SEQUENCE LISTING (14 sequences)

SEQ ID NO: 1 - RNA, 86 nt, Homo sapiens
ugccagucucuaggucccugagacccuuuaaccugugaggacauccagggucacaggugagguucuugggagccuggcgucuggcc

SEQ ID NO: 2 - RNA, 24 nt, Homo sapiens
ucccugagacccuuuaaccuguga

SEQ ID NO: 3 - RNA, 22 nt, Homo sapiens
acaggugagguucuugggagcc

SEQ ID NO: 4 - DNA, 20 bp, Artificial Sequence
  Description of Artificial Sequence Synthetic primer
gaagtcaggtctctaaccag

SEQ ID NO: 5 - DNA, 22 bp, Artificial Sequence
  Description of Artificial Sequence Synthetic primer
cttcaagctcatttctgcacag

SEQ ID NO: 6 - DNA, 20 bp, Artificial Sequence
  Description of Artificial Sequence Synthetic primer
ccaagagttcttgataggag

SEQ ID NO: 7 - DNA, 22 bp, Artificial Sequence
  Description of Artificial Sequence Synthetic primer
cttcaagctcatttctgcacag

SEQ ID NO: 8 - RNA, 23 nt, Artificial Sequence
  Description of Artificial Sequence Synthetic oligonucleotide
cacagguuaaagggucucaggga

SEQ ID NO: 9 - RNA, 22 nt, Artificial Sequence
  Description of Artificial Sequence Synthetic oligonucleotide
cucgcguagaagaguaggugga

SEQ ID NO: 10 - DNA, 27 bp, Artificial Sequence
  Description of Artificial Sequence Synthetic primer
ccgcacaccatgttgccagtctctagg

SEQ ID NO: 11 - DNA, 29 bp, Artificial Sequence
  Description of Artificial Sequence Synthetic primer
cccaggtgtgtggttgggccagacgccag

SEQ ID NO: 12 - DNA, 24 bp, Artificial Sequence
  Description of Artificial Sequence Synthetic oligonucleotide
tcacaggttaaagggtctcaggga

SEQ ID NO: 13 - DNA, 86 bp, Homo sapiens
tgccagtctctaggtccctgagaccctttaacctgtgaggacatccagggtcacaggtgaggttcttgggagcctggcgtctggcc

SEQ ID NO: 14 - DNA, 24 bp, Homo sapiens
tccctgagaccctttaacctgtga

(19) United States
(12) Patent Application Publication
(10) Pub. No.: US 2011/0280861 A1
(43) Pub. Date: Nov. 17, 2011

(54) METHOD FOR MIR-125A IN PROMOTING HEMATOPOIETIC STEM CELL SELF RENEWAL AND EXPANSION

(75) Inventors: David Scadden, Weston, MA (US); Shangqin Guo, North Haven, CT (US); Jun Lu, North Haven, CT (US)

(73) Assignees: MASSACHUSETTS INSTITUTE OF TECHNOLOGY, Cambridge, MA (US); THE GENERAL HOSPITAL CORPORATION, Boston, MA (US)

(21) Appl. No.: 13/058,076

(22) PCT Filed: Aug. 10, 2009
(86) PCT No.: PCT/US09/53290

§ 371 Date: Jun. 7, 2011

Related U.S. Application Data
(60) Provisional application No. 61/087,237, filed on Aug. 8, 2008.

(51) Int. Cl. A61K 31/7105; A61K 31/7088; A61K 39/395; A61P 35/02; A61K 35/12; A61P 37/02; A61P 35/00; C12N 5/0789; A61K 38/02

(52) U.S. Cl. 424/130.1; 435/325; 435/374; 514/44 R; 514/1.1; 424/93.7

(57) ABSTRACT

Embodiments of the invention relate to methods and compositions for the expansion of hematopoietic stem cell (HSC) self renewal. The microRNA-125a is a master control of HSC self-renewal. Increased expression of mir-125a increased HSC self-renewal by 6-30 folds. Increased expression of mir-125a can be used to expand HSC ex vivo and in vivo.

### CROSS REFERENCE TO RELATED APPLICATION

This application claims benefit under 35 U.S.C. §119(e) of the U.S. Provisional Application No. 61/087,237 filed Aug. 8, 2008, the contents of which are incorporated herein by reference in its entirety.

### GOVERNMENT SUPPORT

This invention was made with Government support under contract No. U54 HL081030 and T32 CA073479 awarded by the National Institute of Health. The Government has certain rights in the invention.

### BACKGROUND OF INVENTION

Hematopoietic stem cells (HSCs) are stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells).

As stem cells, they are defined by their ability to form multiple cell types (multipotency) and their ability to self-renew. It is known that a small number of HSCs can expand to generate a very large number of progeny HSCs. This phenomenon is used in bone marrow transplant when a small number of HSCs reconstitute the hematopoietic system. The ability to self-renew and differentiate to form all blood cells provides a constant supply of blood cells throughout animal life. The supply is need for replacing old, worn out or dead blood cells in the body.

However, there are instances when HSC self renewal is or becomes severely impaired due to genetic disorders, diseases, illness, medical treatment and/or environmental effects. The affected individual cannot supply blood cells for replacing old, worn out or dead blood cells in the body, becomes deficient in life sustaining blood cells, consequently becomes susceptible to infections and has to be dependent on a life time of blood transfusion for the replacement of blood cells in the body. There is a need for new innovations that can boost or recover HSC self renewal capability.

### SUMMARY OF THE INVENTION

Embodiments of the present invention are based on the discovery that a mircoRNA, miR-125a, is a positive regulator of hematopoietic stem cell (HSC) expansion and self renewal. Expression of miR-125a as a transgene is necessary and sufficient to expand the numbers HSC cell division by at least 10 fold without a loss of multi-lineage potential in the expanded progeny HSCs. However, in more mature cells with a committed lineage, miR-125a transgene expression is insufficient to induce self-renewal.

Accordingly, an embodiment of the present invention provides a method of expanding HSC production in a subject in need thereof, the method comprising providing a therapeutically effective amount of a nucleic acid sequence comprising miR-125a to the subject, thereby expanding HSC production in the subject. The nucleic acid sequence can comprise, e.g., SEQ. ID. No. 1 or 2. In one embodiment, the nucleic acid comprises at least 90% identical to SEQ. ID. No. 1 or 2. In other embodiments, the nucleic acid is at least 92%, at least 93% at least 94%, at least 95%, at least 96%, at least 97%, at least 98%, at least 99%, and all the intermediate percentages between 90% and 100%, identical to SEQ. ID. No. 1 or 2. Any differences from SEQ. ID. No. 1 should be such that the overall stem loop hairpin structure of the pri-miR-125a is maintained.

In one embodiment, the nucleic acid sequence comprising miR-125a is expressed from a vector containing a nucleic acid sequence encoding miR-125a or a precursor thereof. The vector can be a virus or a non-virus. The vector can be selected from a plasmid, cosmid, phagemid, or virus. In one embodiment, the vector further comprises one or more in vivo expression elements operatively linked to the nucleic acid sequence encoding miR-125a or a precursor thereof for expression in HSCs, such as promoter or enhancer and combinations thereof.

In one embodiment, encompassed in the method of expanding HSC production, the therapeutically effective amount of a nucleic acid sequence comprising miR-125a is provided by administering a pharmaceutical composition comprising i) a nucleic acid sequence comprising miR-125a or ii) a vector expressing a nucleic acid sequence encoding miR-125a or a precursor thereof.

In one embodiment, the subject is a mammal. In another embodiment, the mammal is a human.

In some embodiments, the subject has received, will receive or is concurrently receiving chemotherapy or radiation therapy. In other embodiments, the subject has received, will receive or is concurrently receiving granulocyte colony-stimulating factor (G-CSF).

In some embodiments, the subject has a disorder selected from the group consisting of myeloma, non-Hodgkin's lymphoma, Hodgkins lyphoma and leukaemia. In other embodiments, the subject has a disorder characterized by a lack of functional blood cells such as a platelet deficiency, neutropenia or anemia, aplastic anemia, sickle cell anemia, fanconi's anemia and/or acute lymphocytic anemia.

In some aspects, the subject has a disorder characterized by a lack of functional immune cells, wherein the immune cells are T or B lymphocytes and the disorder is selected from the group consisting of lymphocytopenia, lymphorrhea, lymphostasis and AIDS.

In other aspects, the subject has received, will receive or is receiving an immuno-suppressive drug. In another aspect, the subject is a stem cell donor.

Provided herein is a method of expanding ex vivo a population of HSCs. In one embodiment, the method comprises contacting a HSC with a nucleic acid sequence comprising miR-125a, thereby expanding ex vivo a population of HSCs. The nucleic acid sequence can comprise, for example, SEQ. ID. No. 1 or 2. In one embodiment, the nucleic acid is at least 90% identical to SEQ. ID. No. 1 or 2. In other embodiments, the nucleic acid is at least 92%, at least 93% at least 94%, at least 95%, at least 96%, at least 97%, at least 98%, at least 99%, and all the intermediate percentages between 90% and 100%, identical to SEQ. ID. No. 1 or 2. Any differences from SEQ. ID. No. 1 should be such that the overall stem loop hairpin structure of the pri-miR-125a is maintained.

In one embodiment, the nucleic acid sequence comprising miR-125a is expressed from a vector containing a nucleic acid sequence encoding miR-125a or a precursor thereof. The vector can be a viral or a non-viral vector. The vector can be selected from a plasmid, cosmid, phagemid, or virus. In one embodiment, the vector further comprises one or more expression elements operatively linked to the nucleic acid sequence encoding miR-125a or a precursor thereof for expression in HSCs, such as promoter or enhancer and combinations thereof.

In one embodiment, the method of expanding ex vivo a population of HSC, further comprises expanding the HSCs for at least one cell doubling ex vivo.

In another embodiment, the method further comprises cryopreserving the expanded HSCs. The expanded HSCs can be used in therapeutically in a subject, e. g. after cancer treatment.

In another embodiment, provided herein is a method of expanding HSC production in a subject in need thereof, the method comprising providing a therapeutically effective amount of an agent that increases the expression miR-125a to the subject, thereby expanding HSC production in the subject. In one embodiment, the agent is selected from a group consisting of a small molecule, nucleic acid, nucleic acid analogue, protein, antibody, peptide, aptamer and variants or fragments thereof that increase expression of miR-125a. Such an agent can take the form of any entity which is normally not present or not present at the levels being administered to the cell or organism.

In one embodiment, the agent is administered to the subject in a pharmaceutical composition comprising the agent and a pharmaceutically acceptable carrier. The pharmaceutical composition can be administered to the subject together with additional therapeutic agents, cancer therapy, immunosuppressant therapy, immunodeficiency therapy, steroid therapy, and psychotherapy.

### BRIEF DESCRIPTION OF THE DRAWINGS

FIG. 1A shows the distribution of hematopoietic-derived nuclear lineages and platelets one day after full dose of pIpC in the donor bone marrow transplant experiment.

FIG. 1B shows total donor type cells in peripheral blood after treatment with pIpC in the donor bone marrow transplant experiment.

FIG. 1C shows B220+, CD3+ and Mac1+ donor type cells in peripheral blood after treatment with plpC in the donor bone marrow transplant experiment.

FIG. 1D shows that the donor cell contribution to immuno-phenotypic stem cells are reduced in the donor bone marrow transplant experiment.

FIG. 2A shows the genomic DNA of donor type cells from the peripheral blood that were FACS sorted 6 months post-transplant.

FIG. 2B shows the colonies of donor type cells with various genotype amplified on methylcellulose media.

FIG. 2C shows the genomic DNA of cells treated with and without interferon beta.

FIG. 2D shows the effects of Dicer loss on number of viable cells among different primitive hematopoietic populations.

FIG. 2E shows the increased apoptosis seen in mutant bone marrow population after Dicer loss. There is increased caspase-3 (activated) in mutant Lin-Kit+Sca+ compartment. Intra-cellular flow cytometry for activated caspase-3. The rectangles in the bottom panel indicate the gates applied to select the Lin-c-Kit+Sca+ (LKS), Lin-c-Kit+Sca− (LK+S−) and Lin-c-Kit-Sca+ (LK-S+) populations.

FIG. 2F shows the increased proliferation was seen in mutant bone marrow following Dicer deletion. Intra-cellular flow cytometry for Ki67 was performed among different hematopoietic compartments.

FIG. 3A shows the global microRNA expression of primitive hematopoietic progenitor compartments with varying degree of self-renewal ability.

FIG. 3B shows the total percentage of GFP expressing bone marrow cells forced to expressed the ˜1 kb cluster carrying the three evolutionary conserved miRNAs that scored high in LT-HSCs display, miR-125a, miR-99b and let-70.

FIG. 3C shows the lineages among GFP expressing bone marrow cells forced to expressed the ˜1 kb cluster carrying the three evolutionary conserved miRNAs that scored high in LT-HSCs display, miR-125a, miR-99b and let-70.

FIG. 3D shows preferential expression of miR-125a, miR-99b and let-7e in long term HSCs as compared to other expression.

FIG. 4A shows the FACS of cells showing the effects of miR-125a, miR-99b or let-70 expression on long term expansion.

FIG. 4B shows the total percentage of GFP expressing HSC cells expressing miR-125a, miR-99b or let-70.

FIG. 4C shows the total percentage of GFP expressing HSC cells expressing miR-125a.

FIG. 4D shows the long-term expansion of GFP expressing HSC cells expressing miR-125a, miR-99b or let-70.

FIG. 4E shows the differentiated lineages among GFP expressing HSC cells expressing miR-125a, miR-99b or let-70.

FIG. 4F shows the long term expansion of GFP expressing HSC cells expressing miR-125a.

FIG. 4G shows the primary and secondary colonies expansion on methylcellulose assays.

FIG. 4H shows a LDA analysis.

FIG. 4I shows the selective protection against apoptosis by miR-125a in lineage negative cells. Quantification is shown on the right. n=5 each. All “*” indicates p<0.05.

FIG. 4J shows that miR-125a targets Bak1.

FIG. 4K shows that miR-125a targets the 3′ UTR of Bak1. Normalized luciferase activities are shown. Error bars represent standard deviation.

FIG. 4L shows that MiR-125a targets Bak1.

FIG. 5A shows the hematopoietic lineages (B220+, CD3+ or Mac1+ cells) prior to and after pIpC injection in the donor bone marrow transplant experiment, in a 1:1 competitive transplantation assay as described in FIG. 1B. C=Controls. M=Mutants.

FIG. 5B are representative FACS plots showing the decreased distribution of Mac1+/CD45.2+ cells after pIpC injection in the donor bone marrow transplant experiment, in primary transplants 6 months post-transplantation. CD45.2+ cells are from Dicer control or mutant donors. Mac1 is a myeloid cell marker.

FIG. 5C are representative FACS plots showing decreased donor-type cell engraftment in secondary transplants 3months post-transplantation.

FIG. 6 shows the genomic DNA of progeny lox/lox and lox/wt cells after increasing dose of interferon beta.

FIG. 7 shows the FACS of cells demonstrating the effects of miR-125a on long term expansion.

### DETAILED DESCRIPTION OF THE INVENTION

Unless otherwise explained, all technical and scientific terms used herein have the same meaning as commonly understood by one of ordinary skill in the art to which this disclosure belongs. Definitions of common terms in hematology and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology can be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Methods in Enzymology: Guide to Molecular Cloning Techniques Vol. 152, S. L. Berger and A. R. Kimmerl Eds., Academic Press Inc., San Diego, USA (1987)); Current Protocols in Molecular Biology (CPMB) (Fred M. Ausubel, et al. ed., John Wiley and Sons, Inc.); Current Protocols in Protein Science (CPPS) (John E. Coligan, et. al., ed., John Wiley and Sons, Inc.); Current Protocols in Immunology (CPI) (John E. Coligan, et. al., ed. John Wiley and Sons, Inc.); Current Protocols in Cell Biology (CPCB) (Juan S. Bonifacino et. al. ed., John Wiley and Sons, Inc.); Culture of Animal Cells: A Manual of Basic Technique by R. Ian Freshney, Publisher: Wiley-Liss; 5th edition (2005); Animal Cell Culture Methods (Methods in Cell Biology, Vol. 57, Jennie P. Mather and David Barnes editors, Academic Press, 1st edition, 1998) which are all incorporated by reference herein in their entirety.

Methods for the production of antibodies are disclosed in PCT publication WO 97/40072 or U. S. Application No. 2002/0182702, which are herein incorporated by reference herein in their entirety. The processes of immunization to elicit antibody production in a mammal, the generation of hybridomas to produce monoclonal antibodies, and the purification of antibodies may be performed as described in “Current Protocols in Immunology” (CPI) (John Wiley and Sons, Inc.); Antibodies: A Laboratory Manual (Ed Harlow and David Lane editors, Cold Spring Harbor Laboratory Press 1988); and Brown, “Clinical Use of Monoclonal Antibodies, ” in Biotechnology and Pharmacy 227-49, Pezzuto et al. (eds.) (Chapman & Hall 1993), all of which are both incorporated by reference herein in their entirety.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Definitions of Terms

As used herein, the term “therapeutically effective amount” refers to an amount of a nucleic acid comprising miR-125a or precursor thereof or an agent that is sufficient to effect a significant increase in the expansion and/or self renewal capability of HSCs. A significant increase is at least 10% greater self renewal capability over that in the absence of the nucleic acid or agent.

As used herein, the term “complementary base pair” refers to A:T and G:C in DNA and A:U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

As used herein, the term “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid sequence is a DNA. In another aspect, the nucleic acid sequence is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

The terms “identical” or percent “identity”, in the context of two or more nucleic acids sequences, refers to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence comprising the mir-125a sequence described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. The BLAST or BLAST 2.0 sequence comparison/alignment algorithms can account for gaps and the like. Preferably, identity exists over a region that is at least about 25 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Nat'l. Acad. Sci. USA 85: 2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

Programs for searching for alignments are well known in the art, e.g., BLAST and the like. For example, if the target species is human, a source of such amino acid sequences or gene sequences (germline or rearranged antibody sequences) can be found in any suitable reference database such as GENBANK™, the NCBI protein databank, VBASE, a database of human antibody genes, and the Kabat database of immunoglobulins or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species can be other than human.

The term “vector”, as used herein, refers to a nucleic acid construct designed for delivery to a host cell or transfer between different host cells. As used herein, a vector can be viral or non-viral. A “vector” also includes a cosmid, phagmid, plasmid or a virus.

As used herein, the term “expression vector” refers to a vector that has the ability to express heterologous nucleic acid fragments in a cell. An expression vector can comprise additional elements, for example, the expression vector can have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the miR-125a gene in place of non-essential viral genes. The vector and/or particle can be utilized for the purpose of transferring any nucleic acids into cells either in vitro or in vivo. Numerous forms of viral vectors are known in the art.

The term “replication incompetent” as used herein means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins from packaging the virus) and viral particles cannot be formed in the patient's cells.

The term “gene” means the nucleic acid sequence (DNA) which is transcribed to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “subject” as used herein includes, without limitation, a human, mouse, rat, guinea pig, dog, cat, horse, cow, pig, monkey, chimpanzee, baboon, or rhesus. In one embodiment, the subject is a mammal. In another embodiment, the subject is a human.

As used herein, the term “hematopoietic stem cell” refers to stem cells that can differentiate into the hematopoietic lineage and give rise to all blood cell types such as white blood cells and red blood cells; all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). “Stem cells” are defined by their ability to form multiple cell types (multipotency) and their ability to self-renew.

As used herein, the term “microRNA” or “miRNA” refers to a microRNA molecule found in eukaryotes that is involved in RNA-based gene regulation. See, e.g., Carrington and Ambros, 2003, Science, 301(5631):336-8 which is hereby incorporated by reference in its entirety. miRNA are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. The term will be used to refer to the RNA molecule processed from a precursor pre-miRNA.

As used herein, the term “miRNA precursor” refers to pri-miRNA and pre-miRNAs.

As used herein, the term “operably linked” refers to that the regulatory elements in the nucleic acid construct are positioned with respect to the miRNA gene such that the miRNA is transcribed to a primary transcript. The regulatory elements include, e.g. promoters for the respective RNA polymerase docking and initiation of transcription.

As used herein, the term “pharmaceutical composition” refers to an active agent in combination with a pharmaceutically acceptable carrier of chemicals and compounds commonly used in the pharmaceutical industry. The term “pharmaceutically acceptable carriers” excludes tissue culture medium.

As used herein, the term “comprising” means that other elements can also be present in addition to the defined elements presented. The use of “comprising” indicates inclusion rather than limitation.

As used herein the term “consisting essentially ” or “ consists essentially” refers to those elements required for a given embodiment. The term permits the presence of elements that do not materially affect the basic and novel or functional characteristic(s) of that embodiment of the invention.

The term “consisting of” refers to methods, and respective components thereof as described herein, which are exclusive of any element not recited in that description of the embodiment.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

Embodiments of the present invention are based on the discovery that a microRNA, miR-125a, is a positive regulator of HSC expansion and self renewal. Expression of miR-125a, as a transgene, is necessary and sufficient to expand the numbers of HSC cell division by at least 10 fold without any loss of multi-lineage differentiation potential in the progenies of the expansion. However, in the more mature, lineage committed cells, expression of miR-125a alone is not sufficient to induce self-renewal in these cells.

MicroRNAs are known to influence lineage fate in hematopoiesis, however little is known about their participation in the stem cell state. Here the inventors report that conditional deletion of the small RNA processing enzyme, Dicer, in adult hematopoietic tissues markedly affected the multipotent primitive population in a cell autonomous manner. Among closely related primitive hematopoietic cells, the stem cell enriched compartment displayed distinctive, exquisite sensitivity to Dicer loss. The inventors identified multiple differentially expressed microRNAs including miR-99b, let-7e and miR-125a that are highly conserved and organize as a 620 base-pair (bp) cluster on mouse chromosome 17. This cluster increased the reconstituting capacity of bone marrow, an effect that could be recapitulated by miR-125a alone. MiR125a increased stem cell number and increased the reconstitution ability of stem cells by ˜10-fold without differentiation blockade. However, miR-125a was not sufficient to induce self-renewal in non-stem cells. Taken together, these data indicate differentiation stage specific dependence for microRNA processing to sustain cell persistence in hematopoiesis and cell state specific capacity for specific microRNA to amplify self-renewal.

HSC self-renew and differentiate to form all blood cells throughout animal life. Adult tissues are comprised of a large number of terminally differentiated effector cells and a much smaller number of long-lived progenitor/stem cells that constantly replenish the pool of the former to maintain tissue homeostasis and repair tissue injury. MicroRNAs, emerging as a class of important cellular regulators, are gaining appreciation as mediators of cell state with specific patterns of microRNA expression demarcating developmental or differentiation stage. These small non-coding RNAs are transcribed by RNA polymerase II and processed by RNases Drosha and Dicer to the mature ˜22 nucleotide (nt) form. In the blood system, multiple microRNAs have been found to direct the differentiation of each of the major blood lineages, e.g. miR-181 for T cell differentiation, miR-150 for B cell maturation and miR-223 for granulopoiesis. MiR-150, in particular, has been shown to shunt the fate choice of megakaryocyte and erythrocyte progenitors (MEP) toward megakaryocytes and away from erythrocytes. Self-renewal can be viewed as one of the unique fate options possessed by stem cells, occupying the very early stages in the blood development program.

Disrupting Dicer in developing mouse embryos causes early lethality with an absence of pluripotent stem cells in the embryo remnants. To assess the role of Dicer in maintaining adult HSCs, the inventors utilized a conditional Dicer allele where the Dicer gene can be efficiently inactivated in the hematopoietic tissue. Specifically, a single microRNA within this cluster, miR-125a, positively regulates the self-renewal ability of HSCs, but is insufficient to induce self-renewal in more mature cells.

MicroRNAs (miRNAs) are a class of 18-24 nt non-coding RNAs (ncRNAs) that exist in a variety of organisms, including mammals, and are conserved in evolution. miRNAs are transcribed as 5′-capped large polyadenylated transcripts (pri-miRNA), primarily in a Pol II-dependent manner. Approximately 40% of human miRNAs are co-transcribed as clusters encoding up to eight distinct miRNA sequences in a single pri-microRNA transcript. Many miRNAs can be encoded in intergenic regions, hosted within introns of pre-mRNAs or within ncRNA genes. Many miRNAs also tend to be clustered and transcribed as polycistrons and often have similar spatial temporal expression patterns. MiRNAs have been found to have roles in a variety of biological processes including developmental timing, differentiation, apoptosis, cell proliferation, organ development, and metabolism (Kloosterman, et al., 2006, Dev. Cell 11:441-50, and Krutzfeldt, et al., 2006, Cell. Metab. 4:9-12). Furthermore, miRNAs have been implicated in diseases such as cancer (Esquela-Kerscher, et al., 2006, Nat. Rev. Cancer 6:259-69) and hepatitis C (Jopling, et al., 2005, Science, 309:1577-81), which make them attractive new drug targets. In contrast to the widely used RNAi technology using small interfering RNA (siRNA) duplexes, strategies to inhibit miRNAs have been less well investigated. Reverse-complement 2′-O-methyl sugar modified RNA is frequently being used to block miRNA function in cell-based systems (Krutzfeldt, et al., 2006, Nat. Genet. 38:S14-9).

Pri-miRNAs are cleaved within the nucleus by the microprocessor complex consisting of Drosha, an RNaseIII-type nuclease and a protein co-factor, DGCR8 (DiGeorge syndrome critical region 8 gene) in humans, Pasha in Drosophila. The resulting 60-70 nucleotide hairpin structure (pre-miRNA) encodes for a single miRNA sequence that is exported from the nucleus to the cytoplasm by Exportin5 in a Ran-GTP dependent manner. Cytoplasmic pre-miRNAs are further cleaved, by another RNaseIII-nuclease, Dicer in concert with cofactors (TRBP and PACT in humans), to remove the loop sequence forming a short-lived asymmetric duplex intermediate (miRNA: miRNA*). The microRNA: microRNA* duplex is in turn loaded into the miRISC complex in which Argonaut (Ago) proteins appear to be the key effector molecules. The strand that becomes the active mature microRNA appears to be dependent upon which has the lowest free energy 5′ end and the other strand is degraded by an unknown nuclease.

Accordingly, in one embodiment, disclosed herein is a method of expanding HSC production in a subject in need thereof, the method comprises providing a therapeutically effective amount of a nucleic acid sequence comprising miR-125a to the subject, thereby expanding HSC production in the subject.

In one embodiment, the nucleic acid sequence comprises SEQ. ID. No. 1 or 2. In other embodiment, the nucleic acid sequence consists of SEQ. ID. No. 1 or 2. In other embodiment, the nucleic acid sequence consists essentially of SEQ. ID. No. 1 or 2. In another embodiment, the nucleic acid sequence is at least 90% identical to SEQ. ID. No. 1 or 2. In one aspect, the nucleic acid is at least 92%, at least 94%, at least 95%, at least 97%, at least 99%, and all the intermediate percentages between 90% and 100%, identical to SEQ. ID. No. 1 or 2. The differences from SEQ. ID. No. 1 or 2 should be such that the overall stem loop hairpin structure of the pri-miR-125a and/or pre-miR-125a is maintained. While SEQ. ID. No. 1 and 2 contain uridine, it is contemplated that the nucleic acid encompassed herein can have thymidine in place of uridine for a DNA nucleic acid sequence.

SEQ. ID. No. 1 is the Homo sapiens, pre-miR-125a having the stem-loop sequence 5′-UGCCAGUCUCUAGGUCCCUGAGACCCUUUAACCUGUGAGGACAUCCAGGGUCACAGGUGAGGUUCUUGGGAGCCUGGCGUCUGGCC-3′. The MiRBase Accession No. is MI0000469 at the World Wide Web MicroRNA Sanger UK site, the ID is hsa-mir-125a, and the symbol is HGNC:MIRN125A. In one embodiment, the thymidine-version of hsa-mir-125a is 5′-TGCCAGTCTCTAGGTCCCTGAGACCCTTTAACCTGTGAGGACATCCAGGGTCACAGGTGAGGTTCTTGGGAGCCTGGCGTCTGGCC-3′ (SEQ. ID. No. 13).

SEQ. ID. No. 2 is the Homo sapiens mature miR-125a sequence 5′-UCCCUGAGACCCUUUAACCUGUGA-3′. The MiRBase Accession No. is MIMAT0000443 and the ID is hsa-miR-125a-5p. In one embodiment, the thymidine-version of mir-125a is 5′-TCCCTGAGACCCTTTAACCTGTGA-3′ (SEQ. ID. No. 14).

In one embodiment, the nucleic acid sequence comprising miR-125a is expressed from a vector containing a nucleic acid sequence encoding miR-125a or a precursor thereof, such as a pri-miR-125a or a pre-miR-125a. The vector can be a virus or a non-virus. The vector can be selected from a plasmid, cosmid, phagemid, or virus. These vectors further comprise one or more in vivo expression elements operatively linked to the nucleic acid sequence encoding miR-125a or a precursor thereof for expression in HSCs, such as promoter or enhancer and combinations thereof. Examples of expression elements operatively linked to the nucleic acid sequence encoding miR-125a include but not limited to the CMV promoter, the SV40 promoter, an inducible promoter such as the tet-repressor (inducible by tetracycline or its derivative doxycycline), the TREX promoter (two Tet-repressor binding sites downstream of CMV's TATA box), and TATA boxes.

In one embodiment, the method includes providing a therapeutically effective amount of a nucleic acid sequence comprising miR-125a to the subject, wherein the therapeutically effective amount of a nucleic acid sequence comprising miR-125a is provided by administering a pharmaceutical composition comprising i) a nucleic acid sequence comprising miR-125a or ii) a vector expressing a nucleic acid sequence encoding miR-125a or a precursor thereof, such as a pri-miR-125a or a pre-miR-125a. In one embodiment, the nucleic acid sequence comprises SEQ. ID. No. 1 or 2.

In some embodiment, the invention provides a pharmaceutical composition comprising a nucleic acid sequence comprising miR-125a. In one embodiment, the miR-125a is either SEQ. ID. No. 1 or 2

In other embodiments, the nucleic acid sequence is at least 90% identical to SEQ. ID. No. 1 or 2. In other aspects, the nucleic acid is at least 92%, at least 94%, at least 95%, at least 97%, at least 99%, and all the intermediate percentages between 90% and 100%, identical to SEQ. ID. No. 1 or 2. Any differences from SEQ. ID. No. 1 or 2 should be such that the overall stem loop hairpin structure of the pri-miR-125a and/or pre-miR-125a is maintained. While SEQ. ID. No. 1 and 2 contain uridines; it is contemplated that the nucleic acid encompassed herein can have thymidine in place of uridine for a DNA nucleic acid sequence.

In one embodiment, the nucleic acid sequence that is at least 90% identical to SEQ. ID. No. 1 or 2 is amplified and cloned by PCR into a vector. For example, the stem-loop SEQ. ID. No. 1 can be amplified by PCR primers 5′-CCGCACACCATGTTGCCAGTCTCTAGG-3′ (SEQ. ID. No. 10 and 5′-CCCAGGTGTGTGGTTGGGCCAGACGCCAG-3′ SEQ. ID. No. 11) using human genomic DNA as template. PCR amplification is well known to one skilled in the art. The amplified DNA can be cloned directly into an expression vector by way of additional restriction enzyme sites engineered into the PCR primers. Examples of suitable expression vectors are the STEMGENT® iPSC generation DOX inducible human TF lentivirar vectors and pseudotyped Human Immunodeficiency virus type 1-derived lentivirus vectors that is replication defective (L G Johnson, et al., 2000, Gene Therapy, 7:568-574. Other commercially available expression vectors for expression in mammalian cells can also be used.

In one embodiment, the vector expressing a nucleic acid sequence encoding miR-125a or a precursor thereof is selected from a plasmid, cosmid, phagemid, or virus.

In another embodiment, the vector further comprises one or more in vivo expression elements operatively linked to the nucleic acid sequence encoding miR-125a or a precursor thereof for expression in HSCs.

In some aspects, the in vivo expression element encompassed within the vector expressing a nucleic acid sequence encoding miR-125a or a precursor thereof comprises a promoter or enhancer and combinations thereof.

In one embodiment, the method of expanding HSC production in a subject in need thereof, the subject is a mammal. In a preferred embodiment, the mammal is a human. It is contemplated that the method described herein is applicable to any mammal having a hematopoietic system and possessing a conserved miR-125a in its genome.

In one embodiment, provided herein is a method of expanding HSC production in a subject in need thereof, the method comprises providing a therapeutically effective amount of an agent that increases the expression miR-125a to the subject, thereby expanding HSC production in the subject.

In one embodiment, the invention provides a method of ex vivo expansion of a production of HSCs, the method comprises contacting an isolated HSC with an agent that increases the expression miR-125a in the cell.

In another embodiment, the therapeutically effective amount of an agent that increases the expression miR-125a to the subject is provided by administering a pharmaceutical composition comprising the agent and a pharmaceutically acceptable carrier.

In some embodiment, the agent is a small molecule, nucleic acid, nucleic acid analogue, protein, antibody, peptide, aptamer and variants or fragments thereof effective to increase miR-125a expression.

In another embodiment, provided herein is a method of expanding ex vivo a population of HSCs, the method comprises contacting an isolated HSC with a nucleic acid sequence comprising miR-125a, thereby expanding ex vivo a population of hematopoietic cells.

In alternative embodiments, provided herein is a method of expanding ex vivo a population of HSCs, the method comprises contacting an isolated HSC with a vector expressing a nucleic acid sequence comprising miR-125a or contacting an isolated HSC with a therapeutically effective amount of an agent that increases the expression miR-125a in the a HSC.

In some aspects, the nucleic acid sequence for expanding ex vivo a population of HSCs is at least 90% identical to SEQ. ID. No. 1 or 2. In other aspects, the nucleic acid is at least 92%, at least 94%, at least 95%, at least 97%, at least 99%, and all the intermediate percentages between 90% and 100%, identical to SEQ. ID. No. 1 or 2. Any differences from SEQ. ID. No. 1 or 2 should be such that the overall stem loop hairpin structure of the pri-miR-125a and/or pre-miR-125a is maintained. While SEQ. ID. No. 1 and 2 contain uridine, it is contemplated that the nucleic acid encompassed herein can have thymidine in place of uridine for a DNA nucleic acid sequence.

In one embodiment, the nucleic acid sequence that is at least 90% identical to SEQ. ID. No. 1 or 2 is amplified and cloned by PCR into a vector. In one embodiment, the vector is an expression vector. Methods of amplifying and cloning DNA sequences are well knows in the art and is also described herein.

In one embodiment, the vector expressing a nucleic acid sequence encoding miR-125a or a precursor thereof is selected from a plasmid, cosmid, phagemid, or virus.

In another embodiment, the vector further comprises one or more expression elements operatively linked to the nucleic acid sequence encoding miR-125a or a precursor thereof for expression in HSCs.

In some aspects, the expression element encompassed within the vector expressing a nucleic acid sequence encoding miR-125a or a precursor thereof comprises a promoter or enhancer and combinations thereof.

In some embodiment, the agent that can increases the expression miR-125a in the HSC ex vivo is a small molecule, nucleic acid, nucleic acid analogue, protein, antibody, peptide, aptamer and variants or fragments thereof effective to increase miR-125a expression.

In some embodiments, the method of expanding ex vivo a population of HSCs described herein further comprises expanding the HSCs for at least one cell doubling ex vivo. The HSCs can be expanded from 1-10 cell doublings or more.

In some embodiments, the method of expanding ex vivo a population of HSCs described herein further comprises cryopreserving the expanded HSCs.

In one embodiment, the expanded HSCs described herein are used therapeutically in a subject, meaning that the expanded HSCs are used to treat deficiencies, genetic defects, diseases, disorders, or ailments associated with the hematopoietic system, e.g. treatment of blood cancers after chemotherapy, treatment of anemia and hemophilia to name a few.

In some embodiments, the method of expanding ex vivo a population of HSCs described herein further comprises introducing the expanded HSCs into a subject in need thereof, such as in the treatment of leukemia or anemia. Where cryopreserved expanded HSCs are used, the frozen cells are thawed and then introduced into the subject. Alternatively, the thawed HSCs can be cultured ex vivo for additional cell doubling to verify viability prior to introduction into the subject.

While not wishing to be bound by theory, methods described herein aim at increasing the expression of miR-125a in HSCs, in vivo or ex vivo, thereby increasing the number or population of non-committed pluripotent HSCs. The aim of increasing the expression of miR-125a in HSCs is achieved by providing an agent that increases the expression miR-125a from the endogenous gene within the subject's genome, or providing a transgene of miR-125a by way of providing a nucleic acid sequence of miR-125a to the HSC or a vector carrying a nucleic acid sequence of miR-125a. The overall anticipated increase in expression of miR-125a in HSCs serves to positively increase the number of times a HSC can self-renew, i. e. cell divide to make more of itself, HSCs that are non-committed to a definite hematopoietic lineage and has retained pluripotent capacity to produce all blood cells.

In many aspects, the methods described herein are applicable to a subject who has reduced capacity or loss of the capability to produce one or more of the blood cells from the hematopoietic lineage, e. g. white blood cells: T or B cells, platelets etc. The subject can be suffering from an illness, a disease, or is being treated for the illness, disease, or a medical or genetic condition, such that the subject has reduced capacity or loss of the capability to produce one or more of the blood cells from the hematopoietic lineage. In some aspects, the subject has received, will receive or is concurrently receiving chemotherapy or radiation therapy. In some aspects, the subject has a disorder selected from the group consisting of myeloma, non-Hodgkin's lymphoma, Hodgkins lyphoma and leukaemia.

In other aspects, the subject has received, will receive or is concurrently receiving G-CSF. In one aspect, the subject has a disorder characterized by a lack of functional blood cells, for example, the disorder is a platelet deficiency or an anemia. Examples of anemia include but are not limited to aplastic anemia, sickle cell anemia, fanconi's anemia and acute lymphocytic anemia.

In another aspect, the disorder is a neutropenia. In one aspect, the subject has a disorder characterized by a lack of functional immune cells, such as T or B lymphocytes. Examples of disorders characterized by a lack of functional immune cells include but are not limited to lymphocytopenia, lymphorrhea, lymphostasis and AIDS.

It is also contemplated that the methods described herein are applicable to subjects who have received, will receive or are receiving an immuno-suppressive drug.

In another embodiment, the methods described herein are applicable to subjects that are stem cell donors, bone marrow donors, and stem cell transplant recipients.

In some embodiments, the ex vivo expanded HSCs are used in hematopoietic stem cell transplantation (HSCT) for the treatment of malignant and nonmalignant disorders, for example but not limited to disorders selected from the list in Table 6. The 2 major types of HSCT have been autologous and allogeneic transplantations, which include the but are not limited to the disorders listed in Table 6.

Autologous transplantation refers to the use of the patient's own stem cells as a rescue therapy after high-dose myeloablative therapy. This is generally used in chemosensitive hematopoietic and solid tumors to eliminate all malignant cells by administering high-dose chemotherapy with subsequent rescue of the host's bone marrow with previously collected autologous stem cells. Immunosuppression is not required after autologous transplantation.

Allogeneic transplantation refers to the use of stem cells from a human leukocyte antigen (HLA)-matched related or unrelated donor. This is used for a variety of malignant and nonmalignant disorders to replace a defective host marrow or immune system with the normal donor marrow and immune system. The key to successful allogeneic transplantation is finding an HLA-matched donor because it decreases the risk of graft rejection and graft versus host disease (GVHD).

The three HLA loci critical for matching are HLA-A, HLA-B, and HLA-DR. HLA-C, and HLA-DQ were recently added to this list. These represent the minimum number of cell surface antigen matching required for transplantation. A completely matched sibling donor is considered ideal. For unrelated donors, a completely matched or a single mismatch is considered acceptable for most transplantation protocols. Syngeneic transplantation is a form of allogeneic transplantation in which the donor is an identical twin sibling of the patient. Graft rejection is less of an issue for such transplants when compared to other allogeneic transplants. The donor's HSCs can be transfected with a vector or nucleic acid comprising the nucleic acid encoding mir-125a that is at least 90% identical to SEQ. ID. No. 1 or 2, ex vivo culture expanded, and then transplanted into the host. Alternatively, the donor's HSCs can be contacted with an agent that increases the expression of the endogenous miR-125a gene, thereby increasing the HSCs capacity to self-renew. These HSCs can then be ex vivo culture expanded, and then transplanted into the host.

TABLE 6
Common indications for HSCT

| Autologous Transplantation | | Allogeneic Transplantation | |
|---|---|---|---|
| Malignant Disorders | Nonmalignant Disorders | Malignant Disorders | Nonmalignant Disorders |
| Multiple | Autoimmune | AML | Aplastic |
| myeloma | disorders | Non-Hodgkin | Anemia |
| Neuroblastoma | Amyloidosis | lymphoma | Fanconi |
| Non-Hodgkin | | Hodgkin | anemia |
| lymphoma | | disease | Severe |
| Hodgkin disease | | Acute | combined |
| Acute myeloid | | lymphoblastic | immunodefi- |
| leukemia (AML) | | leukemia (ALL) | ciency |
| Medulloblastoma | | Chronic | Thalassemia |
| Germ-cell | | myeloid | major |
| tumors | | leukemia | Diamond- |
| | | (CML) | Blackfan |
| | | Myelodysplastic | anemia |
| | | syndromes | Sickle cell |
| | | Multiple | anemia |
| | | myeloma | Wiskott- |
| | | Chronic | Aldrich |
| | | Lymphocytic | Syndrome |
| | | Leukemia | Osteopetrosis |
| | | | Inborn errors |
| | | | of |
| | | | metablolism |
| | | | Autoimmune |
| | | | disorders |

In some embodiment, the administering in the methods described herein is intravenous, intradermal, intramuscular, intraarterial, intralesional, percutaneous, subcutaneous, or by aerosol.

The inventors have shown in the Examples that (i) peripheral HSCs can be isolated from mice; (ii) the isolated HSCs can be transfected with a lentivirus comprising the mir-125a gene; (iii) the transfected HSCs increased 6-30 folds of self-renewal; and (iv) when these transfected HSCs are implanted into mice in a primary and secondary transplantation experiments, these transfected HSCs established and populate the bone marrow.

In one embodiment, the invention also provides a method of inhibiting HSC self-renewal in a subject in need thereof, the method comprises administering an effective amount of an agent that inhibits mir-125a to a subject. In one embodiment, inhibiting HSC self-renewal prevents excessive or deregulated cell proliferation, e. g. in chronic and acute leukemia.

In one embodiment, disclosed herein is a method of treating blood cancer in a subject in need thereof, the method comprising administering an effective amount of an agent that inhibits mir-125a in a cell to a subject. In one embodiment, blood cancer is selected from a group consisting of lymphoma, leukemia and multiple myeloma.

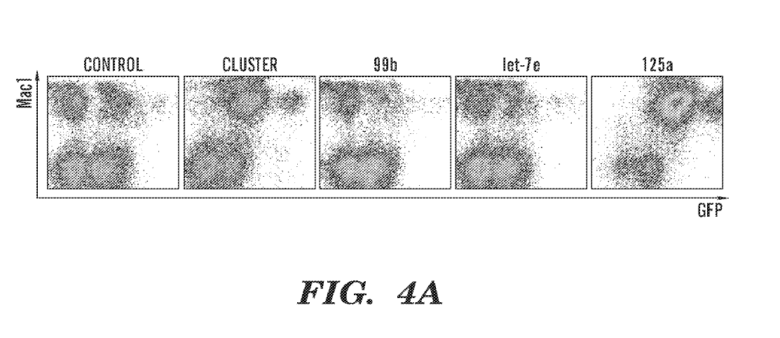

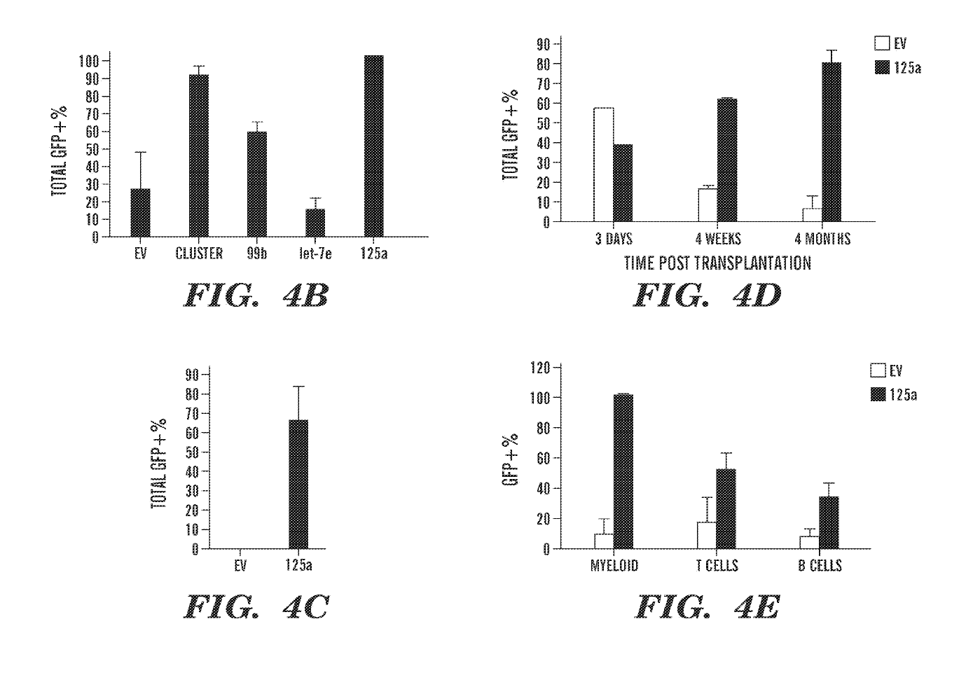

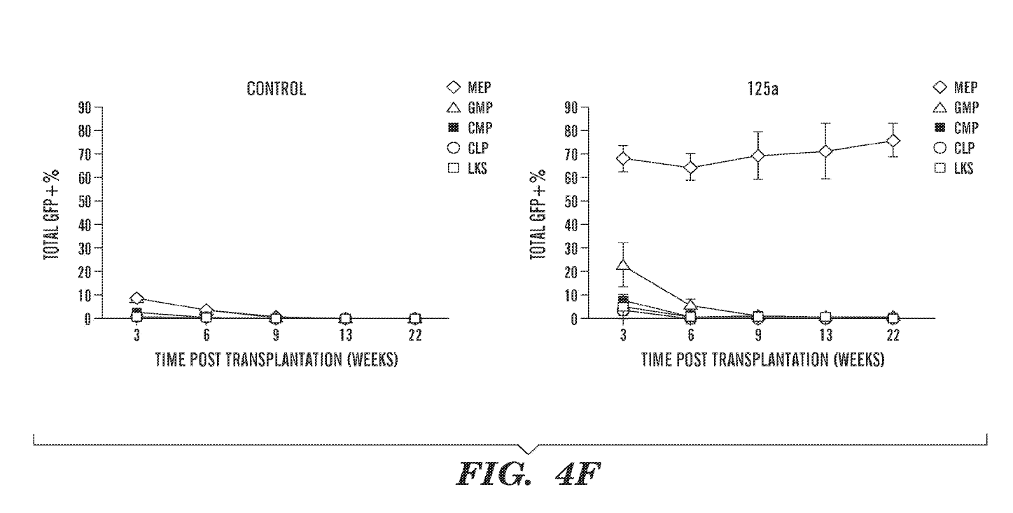

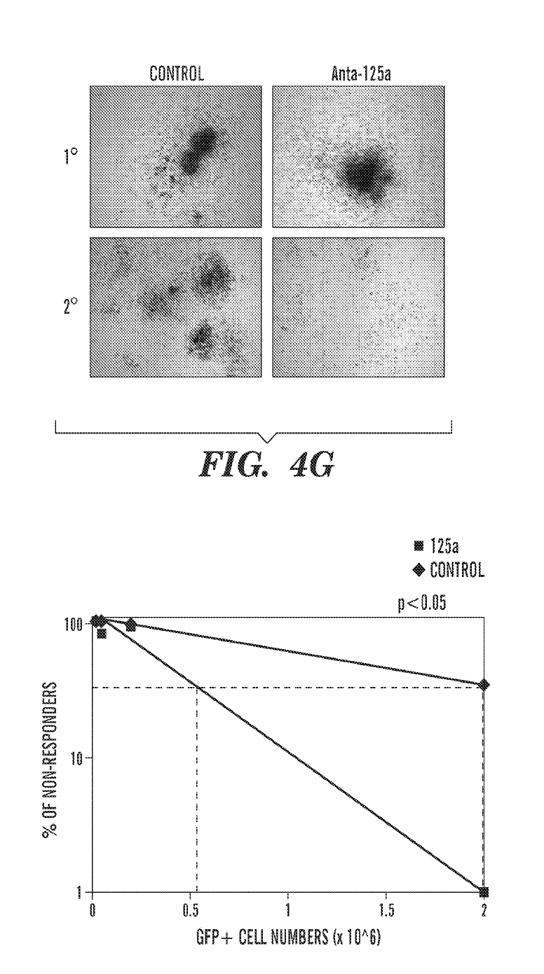

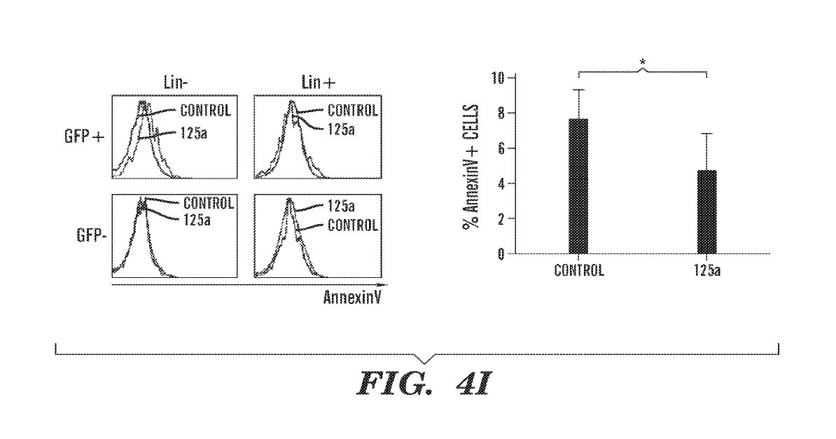

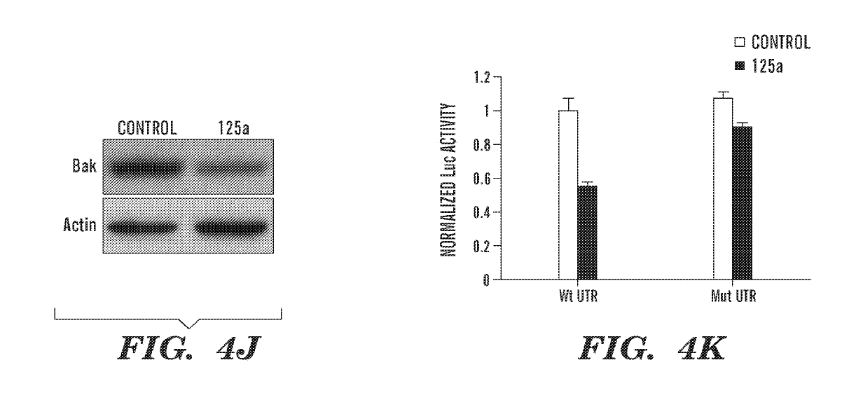

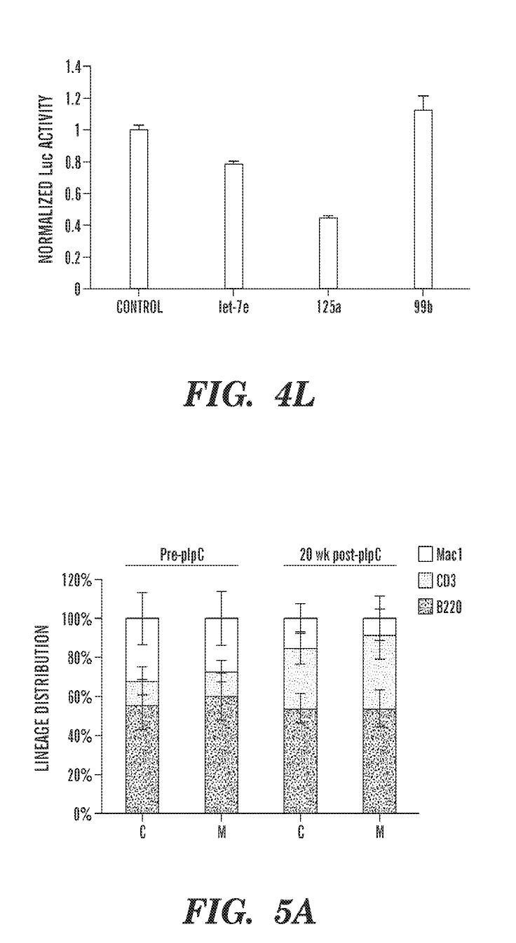

In some aspects, an agent that inhibits mir-125a activity in a cell can be referred to as a mir-125a inhibitor. The mir-125a inhibitor functions by blocking, preventing, and/or antagonizing the normal cellular activity of the mature mir-125a which is to down regulate the expressions of certain genes, which may include suppression of the expression of Bak1, as disclosed herein in the Examples and in FIG. 4J, and other genes such as ERBB2 (HER2) and ERBB3 (HER3), two important tyrosine kinase receptors frequently deregulated in breast cancer, and the truncated Neurotrophin Receptor Tropomyosin-Related Kinase C (NTRK3), a key regulator protein of the neuroblastoma cell proliferation. miR-125a can also target different and suppress expression of different genes in HSC. miR-125a functions as a tumor suppressor and has previously been identified to be downregulated in breast cancer (e.g. in ERBB2-amplified and overexpressing breast cancers). Restoration of miR-125a impaired tumor cell growth and reduced tumor cell migration and invasion, demonstrating miR-125 functions as a tumor suppressor. Ectopic expression of miR-125a in the ERBB2 dependent human breast cancer line, SKBR3, caused suppression of its anchorage-dependent growth and inhibition of its mobility and invasive capabilities. Ectopic expression miR-125a in non-transformed and ERBB2-independent MCF10a cells produced inhibitory effects on its anchorage-dependent growth and no significant impact on the mobility of these non-invasive human breast epithelial cells. Furthermore, miR-125a targets, ERBB2 and ERBB3, were downregulated when miR-125a is expressed in SKBR3 cells, and downregulation of ERBB2 and ERBB3 decreased the motility and invasiveness features of SKBR3 cells.

A mir-125a inhibitor can be an antagomir of mir-125a, an antisense oligonucleotide to mir-125a, a locked nucleic acid that anneals to mir-125a, an siRNA of mir-125a and double-stranded RNA corresponding to mir-125a (dsRNA).

In another embodiment, the agent is a siRNA to the mir-99b-let7e-mir-125a gene cluster in the human genome. In other embodiments, the siRNA is modified. Methods of siRNA modification are well known in the art, e. g. in U.S. published applications Nos. 2005/0233329, 2005/0233342, 2004/0219671, 2004/0266707, 2006/0122137, 2005/0032733, U.S. patent application Ser. Nos. 10/916,185; 10/946,873; 10/985,426; and 10/560,336, all of which are incorporated by reference in their entirety.

In one embodiment, an agent that inhibits mir-125a activity in a cell comprises a nucleic acid sequence that can form complementary base-pairing with SEQ. ID. No. 2, the mature mir-125a, for at least 90% of the bases of SEQ. ID. No. 2. In one aspect, the nucleic acid can form complementary base-pairing with at least 92%, at least 94%, at least 95%, at least 97%, at least 99%, and all the intermediate percentages between 90% and 100%, to SEQ. ID. No. 2. In another embodiment, an agent is a vector comprising a nucleic acid sequence that is at least 90% identical to SEQ. ID. No. 3 (miRBase:MIMAT0004602). The nucleic acid is at least 92%, at least 94%, at least 95%, at least 97%, at least 99%, and all the intermediate percentages between 90% and 100%, identical to SEQ. ID. No. 3. In yet another embodiment, the agent is a nucleic acid sequence that is at least 90% identical to SEQ. ID. No. 3, at least 92%, at least 94%, at least 95%, at least 97%, at least 99%, and all the intermediate percentages between 90% and 100%, to SEQ. ID. No. 3. In other embodiment, the agent is a nucleic acid sequence comprising SEQ. ID. No. 3. In another embodiment, the agent is a nucleic acid sequence consisting of SEQ. ID. No. 3. In yet another embodiment, the agent is a nucleic acid sequence consisting essentially of SEQ. ID. No. 3.

In one embodiment, a mir-125a inhibitor is between 17 and 25 nucleotides in length and that comprises a 5′ to 3′ sequence that is at least 90% complementary to the 5′ to 3′ sequence of SEQ. ID. No. 2. In another embodiment, a mir-125a inhibitor is a synthetic RNA molecule of between 17 and 125 residues in length comprising i) an miRNA region whose sequence from 5′ to 3′ is identical to a mature mir-125a sequence, and ii) a complementary region whose sequence from 5′ to 3′ is between 60% and 100% complementary to the mature mir-125a sequence.

Antagomirs are a novel class of chemically engineered oligonucleotides that block the activity of miRNAs and essentially “silence” the miRNA (Krützfeldt J, et. al., 2005, Nature 438: 685-9). Antagomirs are single-stranded RNA that are perfectly complementary to their miRNA except that they are 2′-O-methyl (2′-OMe) oligoribonucleotides and are also linked to cholesterol at the 3′ end. Both these modifications, 2′-OMe and cholesterol, aid in the antagomir stability in vivo and ease of entry into the cells. Methods of designing and synthesizing antagomirs and the various modifications (e.g. 2′-O-Methoxyethyl) are described in US Pat. Application 20070213292 and is hereby incorporated by reference in its entirety. An example of a mir-125a antagomir is 5′ mC(*)mA(*)mCmAmGmGmUmUmAmAmAmGmGmGmUmCmUmCmAmG(*)mG(*)mG(*)mA(*)(3′-Chl) 3′ (SEQ. ID. No. 8). The mN: 2′OMe base; *: phosphorothioate linkage; Chl: cholesterol.

In one embodiment, the mir-125a inhibitor is a mir-125a antagomir. In one embodiment, the mir-125a inhibitor is SEQ. ID. No. 8. In another embodiment, the mir-125a inhibitor consists essentially of SEQ. ID. No. 8. In another embodiment, the mir-125a inhibitor consists of SEQ. ID. No. 8. In another embodiment, the mir-125a inhibitor comprises SEQ. ID. No. 8.

Locked nucleic acid (LNA)-modified oligonucleotides are distinctive 2′-O-modified RNA in which the 2′-O-oxygen is bridged to the 4′-position via a methylene linker to form a rigid bicycle, locked into a C3′-endo (RNA) sugar conformation (Vester B., et. al., Biochemistry 2004; 43: 13233-13241). The LNA modification leads to the thermodynamically strongest duplex formation with complementary RNA known. Consequently, a biological activity is often attained with very short LNA oligonucleotides. For example, an 8 nt fully-modified LNA oligomer complementary to a structural loop inhibited 50% of self-splicing of group I introns from rRNA genes in pathogenic organisms whereas DNA and RNA oligonucleotides were ineffective. Short fully-modified LNA oligonucleotides designed against telomerase were active in cellular assays, compared to mismatched negative controls. Furthermore, LNAs display excellent mismatch discrimination. Mouritzen et al. (Expert Rev Mol Diagn 2003; 3: 27-38) showed single-nucleotide specificity against complementary DNA using fully modified 12 nucleotide LNA probes coupled to glass slides during the development of a microarray used to probe samples for single-nucleotide polymorphisms (SNPs) associated with human dysmetabolic syndrome. The synthesis and incorporation of LNA bases can be achieved by using standard DNA synthesis chemistry and described in U.S. Pat. No. 6,268,490 and is hereby incorporated by reference in its entirety.

An anti-sense oligonucleotide of mir-125a has a sequence that perfectly complementary to SEQ. ID. No. 2, the mature mir-125a. Complementary pairing between an anti-sense oligonucleotide of mir-125a and mir-125a produces a duplex RNA that is highly susceptible to RNase degradation. An anti-sense oligonucleotide of mir-125a comprises the sequence 5′-TCACAGGTTAAAGGGTCTCAGGGA-3′ (SEQ. ID. No. 12). MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein. In one embodiment, the mir-125a inhibitor is at least 80%, at least 82%, at least 84%, at least 86%, at least 88%, at least 90%, at least 92%, at least 94%, at least 96%, at least 98%, and 100% identical to SEQ. ID. No. 12, including all the intermediate percentages between 80% to 100%.

One skilled in the art can also readily determine an appropriate dosage regimen for administering a compound that inhibits miRNA expression to a given subject, as described herein. Suitable compounds for inhibiting miRNA gene expression include double-stranded RNA (such as short- or small-interfering RNA or “siRNA”), antisense nucleic acids, and enzymatic RNA molecules, such as ribozymes. Each of these compounds can be targeted to a given miRNA gene product and interfere with the expression (e.g., by inhibiting translation, by inducing cleavage and/or degradation) of the target miRNA gene product. For example, expression of a given miRNA gene can be inhibited by inducing RNA interference of the miRNA gene with an isolated double-stranded RNA (“dsRNA”) molecule which has at least 90%, for example at least 95%, at least 98%, at least 99%, or 100%, sequence homology with at least a portion of the miRNA gene product. In a particular embodiment, the dsRNA molecule is a “short or small interfering RNA” or “siRNA.” siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”).The sense strand comprises a nucleic acid sequence that is substantially identical to a nucleic acid sequence contained within the target miRNA gene product.

Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9(1999), and Hermann and Patel, Science 287:820-5 (2000). Aptamers can be RNA or DNA based. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer can be prepared by any known method, including synthetic, recombinant, and purification methods, and can be used alone or in combination with other aptamers specific for the same target. Further, as described more fully herein, the term “aptamer” specifically includes “secondary aptamers” containing a consensus sequence derived from comparing two or more known aptamers to a given target.

In one embodiment, an agent that inhibits mir-125a activity is a vector that comprises an anti-sense oligonucleotide to mir-125a (SEQ. ID. No. 12) or SEQ. ID. No. 3. The anti-sense oligonucleotide sequence or SEQ. ID. No. 3 can be cloned into a vector for the expression in a host cell by any means known to one skilled in the art. In one embodiment, the vector is a virus. In another embodiment, the vector is a non-virus. Designing, cloning, transfection, and expression of anti-sense oligonucleotides against miRNAs are described in Scherr M. et. al., 2007, Nucleic Acid Research 35(22):e149 and is incorporated hereby reference in its entirety.

In one embodiment, the agent can be various combinations of an antagomir of mir-125a, an antisense oligonucleotide to mir-125a, dsRNA to mir-125a, siRNA to mir-125a or a locked nucleic acid that anneals to mir-125a.

In one embodiment, a mir-125 nucleic acid sequence, or agent which increases the expression of miR-125a in a subject, thereby expanding a population of HSC in a subject is targeted to a cell surface marker present on the HSC. In some embodiments, the mir-125 nucleic acid sequence or an agent which increases the expression of miR-125a is attached to a cell surface marker on HSC.

In some embodiments, an agent which inhibits miR-125a (i.e. for the treatment of a blood cancer) is targeted to a HSC, for example, the agent is attached to a moiety which binds to a cell surface marker on a HSC.

Examples of such cell-surface markers for HSC are well known in the art, and include for example, CD31, c-kit and Sca-1, CD34, CD38 and HLA-DR antigens. HSCs are CD34+/CD38−/HLA-DR+. HSCs can also express homogeneous levels of stem cell factor receptor (“SCFR”), Leu 8 (“L-selectin”), CD18, CD33, CD44, CD48, CD49e, CD50 and CD52. Low levels of granulocyte/monocyte-colony stimulating factor receptor (“GM-CSFR”), gp130/IL-6R (gp130 signal subunit of. the interleukin-6 receptor), IL-6R and Fas Ag are expressed on HSCs, while IL-7R is not expressed at all. Hematopoeitic stem cells are identified by their small size, lack of lineage (lin) markers, low staining (side population) with vital dyes such as rhodamine 123 (rhodamineDULL, also called rholo) or Hoechst 33342, and presence of various antigenic markers on their surface, many of which belong to the cluster of differentiation series, like: CD34, CD38, CD90, CD133, CD105, CD45 and also c-kit- the receptor for stem cell factor. The hematopoietic stem cells are negative for the markers that are used for detection of lineage commitment, and are, thus, called Lin-; and, during their purification by FACS, a bunch of up to 14 different mature blood-lineage marker, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. for mice) antibodies are used as a mixture to deplete the lin+cells or late multipotent progenitors (MPP)s. Typical human cell surface HSC markers include CD34+, CD59+, Thy1/CD90+, CD381o/−, C-kit/CD117+, lin-. Cell-surface markers for HSC are also disclosed in U.S. Pat. No. 6,555,324 and US Patent Application 20080076148 which are incorporated herein in their entirety.

In one embodiment, the invention described herein provides a method of screening and identifying agents such as proteins, small molecules, nucleic acids or compounds that can inhibit the mir-125a activity or expression in a cell, the method comprises: (a) contacting an isolated HSC and/or cell expressing the human mir-125a gene with an agent; and (b) determining the mir-125a activity or mir-125a expression level and comparing with reference activity/level of mir-125a in an isolated HSC not in contact with the agent, wherein a decrease in the activity/level of mir-125a indicates that the agent inhibits mir-125a. In one embodiment, the decrease is at least 10% of the reference activity/level, at least 20%, at least 30%, at least 40%, at least 50%, at least 60%, at least 70%, at least 80%, at least 90%, at least 100% of the reference activity/level, including all the intermediate percentages between 10% and 100%.

In one embodiment, the mir-125a activity comprises monitoring its target Bak1. Expression of mir-125a reduces Bak1 protein in the cell. In one embodiment, the mir-125a activity can be monitored by determining the luciferase activity of the Bak1 3′ UTR-luciferase fusion cells disclosed herein.

In one embodiment, method of screening and identifying agents is a high throughput screening.

In some embodiment, small compound libraries can be screened, e. g. the NINDS Custom Collection 2, BIOMOL ICCB Known Bioactives 3, Prestwick1 Collection, BIOMOL ICCB Known Bioactives2, BIOMOL ICCB Known Bioactives1, NINDS Custom Collection, BIOMOL ICCB Known Bioactives1, ICCB Bioactives SpecPlus Collection, Prestwick MMRF collections. In other embodiments, libraries comprising of natural product extracts can be screened. In yet other embodiments, RNAi libraries can be screened. In further embodiments, libraries of peptides and peptidomimetics can be screened. Such libraries are available to the public or are commercially available, e. g. from the Institute of Chemistry and Cell Biology (ICCB) at Harvard Medical School and the Broad Institute at the Massachusetts Institute of Technology. Methods of synthesis of peptides and peptidomimetics are known in the art as described herein.

### Isolation and Expansion of Hematopoietic Stem Cells.

Stem cells can obtained from the bone marrow by repeated aspirations of the posterior iliac crests of a stem donor under general or local anesthesia. A patient's own stem cell can also be collected for ex vivo expansion. Adverse effects are generally rare and include discomfort at the harvesting site that typically lasts 1-2 weeks. This can be a difficult procedure in donors who are smaller than the recipient, such as sibling donors, and several aspirations may be required for an adequate mononuclear cell dose.

Bone marrow primed with granulocyte colony-stimulating factor (G-CSF, filgrastim [NEUPOGEN®]) has been used both in pediatric and adult patients to increase the stem cell count and thus reduce the number of aspirations from the donor and speed engraftment in the recipient. Filgrastim and chemotherapy can be used alone or in combination to mobilize stem cells.

Stem cells in the bone marrow can be mobilized into the peripheral blood and then collected. For patient having undergone a cycle of chemotherapy, they are allowed to recover for a period before stem cells are collected, and their number can be increased by using hematopoietic growth factors like G-CSF. Along with increasing the number of cells, G-CSF also causes the release of proteases that degrade the proteins that anchor the stem cells to the marrow stroma, causing their release into the peripheral blood. Recent literature has shown that combination of G-CSF and AMD3100, an inhibitor of chemokine receptor 4 (CXCR4), is superior to G-CSF alone in mobilizing stem cells.

Alternatively, HSCs can be obtained from circulating blood. Peripheral blood progenitor cells (PBPC) have become the preferred source of hematopoetic stem cells for allogeneic and autologous transplantation because of technical ease of collection and shorter time required for engraftment. As used herein, the term “progenitor” cell refers to an immature or undifferentiated cell, typically found in post-natal animals. Progenitor cells can be unipotent or multipotent. Traditionally, G-CSF has been used to stimulate more PBPC and release of HSCs from the bone marrow. Although regimens using G-CSF usually succeed in collecting adequate numbers of PBPC from healthy donors, 5%-10% will mobilize stem cells poorly and may require multiple large volume apheresis or bone marrow harvesting.

The dosage of G-CSF dose is 5-20 mcg/kg/day. In most regimens, 10 mcg/kg/day is used until harvesting. After mobilization, an apheresis machine collects the cells. Two ports of venous access are necessary to allow for continuous blood processing. In most adults, venous access is accomplished by using 2 antecubital lines. In 5-10% of adults and in most children, percutaneous antecubital large-bore access is not possible, and an apheresis catheter is used instead. Apheresis catheters can be used in children as light as 10 kg. Lighter children generally require a femoral catheter.

The WBC count, or the CD34 count, in the peripheral blood determines the timing of collection. CD34 is a cell surface marker on HSCs. Studies have shown a good correlation between the CD34 count in the peripheral blood and the number of cells harvested. The recommended CD34 count is 20-50 cells/μL of blood.

Collected stem cells are counted by flow cytometric analysis. Although the minimum number required for engraftment is considered to be 1×10⁶ cells per kilogram of body weight, the preferred number is 2-2.5×10⁶ cells/kg. Most people prefer to have a collection goal of 5-10×10⁶ cells/kg to freeze the extra cells for potential future use.

Peripheral-blood stem cells can be cryopreserved for infusion months to years after collection.

AMD3100 is a bicyclam compound that inhibits the binding of stromal cell derived factor-1 (SDF-1) to its cognate receptor CXCR4. CXCR4 is present on CD34+ HSCs and its interaction with SDF-1 plays a pivotal role in the homing of CD34+ cells in the bone marrow. Inhibition of the CXCR4-SDF1 axis by AMD3100 releases CD34+ cells into the circulation, which can then be collected easily by apheresis. Recently, a published report demonstrated that large numbers of CD34+ cells were rapidly mobilized in healthy volunteers following a single subcutaneous injection of AMD3100.

The HSCs can be isolated fresh and then frozen as the mononuclear cells of peripheral blood, cord blood, and bone marrow using its pan-hematopoietic antigen CD34 or by other methods that are known to one skilled in the art. For example, antibodies against CD34 can be used for immuno-isolating the CD34+ HSCs from the mononuclear cell fraction. The anti-CD34 antibodies can be conjugated with fluorophores or to magnetic beads for ease of separation by FACS or magnets respectively.

HSCs bearing the pan-hematopoietic antigen CD34 can also be isolated by taking advantage of the cells' ability to bind galactose-conjugated proteins. This lectin-positive sub-population represents approximately 0.1 to 0.5% of the total bone marrow cells, and contains 100% of the hematopoietic progenitor cells. The galactose-binding lectin on these cells is specific for this sugar. Additionally, highly proliferative HSCs with very primitive phenotypes, including a newly identified progenitor cell that produces multiple lineages, express this lectin. (Pipia and Long, Nature Biotechnology 15, 1007-1011 (1997)).

In vitro transfection of isolated HSCs from a host facilitates targeted transfection of the miR-125a transgene into specific progenitor cells. Transfection of progenitor cells can be accomplished by any transfection methods known in the art, for example, calcium phosphate-mediated, DEAE-Dextran-mediated, calcium alginate microbeads, cation lipid-mediated, liposomes encapsulation, scrape-loading, and ballistic bombardment of nucleic acid gold particles. In one embodiment, isolation and culturing of progenitor cells is performed using the methods well known to those skilled in the art, e. g. as described in U.S. Pat. Nos. 5,199,942, 5,474,687, 5,589,368, 5,612,211, 5,905,041, 6,355,237, and 7,345,025, which are hereby incorporated by reference in their entirety. The identity of the isolated hematopoietic progenitor cells can be confirmed by transglutaminase expression in culture as described in WO2000/006766, which is also hereby incorporated by reference in its entirety. After in vitro transfection, the miR-125a transfection level can be monitored by quantitative real-time PCR with specific primer pairs to the pre-miR-125a and the mature miR-125a. The transfected HSCs carrying the transgene can be expanded in culture according to methods described in U.S. Pat. Nos. 5,744,361, 5,905,041, and 6,326,198, which are hereby incorporated by reference in their entirety. The expanded HSCs with the miR-125a transgene can then be transplanted back into the original host. Transplantation of progenitor cells are described in U.S. Pat. Nos. 5,817,773, 5,858,782, and U.S. patent application Ser. No. 10/730,334 and they are hereby incorporated by reference in their entirety.

In some embodiment, a nucleic acid sequence encoding miR-125a is cloned into an attenuated Vaccinia virus strain MVATGN33; a human Adenovirus serotype 5; an Adeno Associated virus serotype 2; an attenuated Vaccinia virus (Ankara Strain); an attenuated Canarypox Virus (ALVAC); an attenuated Vaccinia Virus (Copenhagen Strain); or an amphotropic murine leukemia virus. The miR-125a carrying viral vectors can be transfected into isolated HSCs. Forty-eight hours after transfection, total RNAs are isolated and loaded onto a 10% denaturing polyacrylamide gel. DNA oligo probes complementary to each of the selected miRNAs are labeled and hybridized to the membrane to detect mature miR-125a that can be efficiently processed (20- to 24-nt). Constructs with high processing efficiency can be selected for therapeutic use in a subject, e. g. in bone marrow transplantation.

In another embodiment, the isolated HSCs are contacted with an agent that increases the expression of the endogenous miR-125a gene, thereby promoting HSC expansion ex vivo. After several cell divisions in ex vivo, the expanded HSCs can be cryopreserved or introduced back into the subject according to methods known to a skilled physician.

The detailed procedure for the isolation of human stem cells is described in Current Protocols in Stem Cell Biology 2007 (Mick Bhatia, et. al., ed., John Wiley and Sons, Inc.) and is hereby incorporated by reference in its entirety.

### Expression Vectors and Expression Systems

Isolated nucleic acid sequences encoding miR-125a that is at least 90% identical to SEQ. ID. No. 1 or 2 can be obtained using a number of standard techniques that are well known in the art. For example, the nucleic acids can be chemically synthesized or recombinantly produced using methods known in the art. In one embodiment, the nucleic acids are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the nucleic acids and their complementary strands can be synthesized as single strand DNA initially and then subsequently anneal together to form DNA duplex for cloning into vectors for gene expression. Restriction enzyme sites can be designed and incorporated at the ends of the eventual duplex to facilitate ligating the duplex into a vector.

Once ligated into a vector, the nucleic acid can be subcloned into any of several expression vectors, such as a viral expression vector or a mammalian expression vector by PCR cloning, restriction digestion followed by ligation, or recombination reaction such as those of the lambda phage-based site-specific recombination using the GATEWAY® LR and BP CLONASE™ enzyme mixtures. Subcloning should be unidirectional such that the 5′ transcription start nucleotide of the nuclei acid sequence is downstream of the promoter in the expression vector. Alternatively, when the nucleic acid sequence is cloned into pENTR/D-TOPO®, pENTR/SD/D-TOPO® (directional entry vectors), or any of the INVITROGEN™'s GATEWAY® Technology pENTR (entry) vectors, the nucleic acid sequence can be transferred into the various GATEWAY® expression vectors (destination) for protein expression in host cells in one single recombination reaction. Some of the GATEWAY® destination vectors are designed for the constructions of baculovirus, adenovirus, adeno-associated virus (AAV), retrovirus, and lentiviruses, which upon infecting their respective host cells, facilitating ease of introducing the transgene into the host cells. The GATEWAY® Technology uses lambda phage-based site-specific recombination instead of restriction endonuclease and ligase to insert a gene of interest into an expression vector. The DNA recombination sequences (attL, attR, attB, and attP) and the LR and BP CLONASE™ enzyme mixtures that mediate the lambda recombination reactions are the foundation of GATEWAY® Technology. Transferring a gene into a destination vector is accomplished in just two steps: Step 1: Clone the nucleic acid sequence of interest into an entry vector such as pENTR/D-TOPO®. Step 2: Mix the entry clone containing the nucleic acid sequence of interest in vitro with the appropriate GATEWAY® expression vector (destination vector) and GATEWAY® LR CLONASE™ enzyme mix. There are GATEWAY® expression vectors for protein expression in E. coli, insect cells, mammalian cells, and yeast. Site-specific recombination between the att sites (attR×attL and attB×attP) generates an expression vector and a by-product. The expression vector contains the nucleic acid sequence of interest recombined into the destination vector backbone. Following transformation and selection in E. coli, the expression vector is ready to be used for expression in the appropriate host.

The nucleic acid sequence of interest can be expressed from recombinant circular or linear DNA vector using any suitable promoter. Suitable promoters for expressing RNA from a vector include, e.g., the U6 or H1 RNA pol III promoter sequences, or the cytomegalovirus promoters. Selection of other suitable promoters is within the skill in the art. The expression vector should have the necessary 5′ upstream and 3′ downstream regulatory elements such as promoter sequences, ribosome recognition and binding TATA box, and 3′ UTR AAUAAA transcription termination sequence for the efficient gene transcription and translation in its respective host cell. The recombinant vectors can also comprise inducible or regulatable promoters for expression of the nucleic acid sequence of interest in hematopoietic progenitor cells. The nucleic acids that are expressed from recombinant vectors can also be delivered and expressed directly in cells. In one embodiment, the nucleic acids are expressed as RNA precursor molecules from a single vector, and the precursor molecules are processed into the functional miR gene product by a suitable processing system, including, but not limited to, processing systems existent within a cell. Other suitable processing systems include, e.g., the in vitro Drosophila cell lysate system (e.g., as described in U. S. Pat. Appl. No. 2002/0086356, and the E. coli RNAse III system (e.g., as described in U. S. Pat. Appl. No. 2004/0014113); the disclosures of which are incorporated herein by reference in their entirety.

Selection of vectors suitable for expressing the nucleic acid sequence, methods for inserting nucleic acid sequences into vector to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee ei al. (2002), Nat. Biotechnol. 20:500-505; and Paul et al. (2002), Nat. Biotechnol. 20:505-508, the entire disclosures of which are incorporated herein by reference.

Examples of expression vectors for mammalian host cells include but are not limited to the strong CMV promoter-based pcDNA3.1 (INVITROGEN™) and pClneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (CLONTECH®), pAd/CMV/V5-DEST, pAd-DEST vector (INVITROGEN™) for adenovirus-mediated gene transfer and expression in mammalian cells; pLNCX2, pLXSN, and pLAPSN retrovirus vectors for use with the Retro-X™ system from CLONTECH® for retroviral-mediated gene transfer and expression in mammalian cells; pLenti4/V5-DEST™, pLenti6/V5-DEST™, pLenti6.2/V5-GW/lacZ (INVITROGEN™), and pPRIME-TREX (Stegmeier F et al. PNAS, USA. 2005, 102:13212-7; Addgene plasmid 11667) for lentivirus-mediated gene transfer and expression in mammalian cells; adenovirus-associated virus expression vectors such as pAAV-MCS, pAAV-IRES-hrGFP, and pAAV-RC vector (STRATAGENE®) for adeno-associated virus-mediated gene transfer and expression in mammalian cells;

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of STRATAGENE®'s ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (El-transformed human embryonic kidney cells) or 911 (E1-transformed human embryonic retinal cells) (Human Gene Therapy 7:215-222, 1996). Recombinant adenoviruses are generated within the HEK 293 cells.

In one embodiment, a recombinant lentivirus can be used for the delivery and expression of a nucleic acid sequence encoding miR-125a that is at least 90% identical to SEQ. ID. No. 1 or 2 in either dividing or non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with VIRAPOWER™ Lentiviral Expression systems from INVITROGEN™.

In one embodiment, a recombinant adeno-associated virus (rAAV) vector can be used for the expression of a nucleic acid sequence encoding miR-125a that is at least 90% identical to SEQ. ID. No. 1 or 2. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particles/ml, are easily obtained in the supernatant and 10¹¹-10¹² viral particles/ml with further concentration. The transgene is integrated into the host genome so expression is long term and stable.

The use of alternative AAV serotypes other than AAV-2 are also known in the art (Davidson et al (2000), PNAS 97(7)3428-32; Passini et al (2003), J. Virol 77(12):7034-40).

Large scale preparation of AAV vectors is made by a three-plasmid cotransfection of a packaging cell line: AAV vector carrying the chimeric DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12;71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsC1 gradients. Delivery vectors can also include but are not limited to replication-defective adenoviral vectors, cationic liposomes and protein-cationic peptides. For example, one study reports a system to deliver DNA in vitro by covalently attaching the surfactant associated protein B (SP-B) to a 10 kDa poly-lysine. See, Baatz, J., et al., PNAS USA, 91:2547-2551 (1994). See, e.g., Longmuir, et al., 1992 ASBMB/Biophysical Society abstract; Longmuir, et al., 1993 Biophysical Society abstract.

### Cryopreservation of Cells

As used herein, “cryopreservation” refers to the preservation of cells by cooling to low sub-zero temperatures, such as (typically) 77 K or −196° C. (the boiling point of liquid nitrogen). Cryopreservation also refers to storing the cells at a temperature between 0°-10° C. in the absence of any cryopreservative agents. At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature.

In one embodiment, the invention provides a cryopreserved pharmaceutical composition comprising: (a) a viable composition of a population of HSCs that has been expanded ex vivo; (for example, as disclosed herein by the methods of paragraphs L to Q) (b) an amount of cryopreservative sufficient for the cryopreservation of the expanded HSCs; and (c) a pharmaceutically acceptable carrier.

Freezing is destructive to most living cells. Upon cooling, as the external medium freezes, cells equilibrate by losing water, thus increasing intracellular solute concentration. Below about 10°-15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotection by solute addition is thought to occur by two potential mechanisms: colligatively, by penetration into the cell, reducing the amount of ice formed; or kinetically, by decreasing the rate of water flow out of the cell in response to a decreased vapor pressure of external ice (Meryman, H. T., et al., 1977, Cryobiology 14:287-302). Different optimal cooling rates have been described for different cells. Various groups have looked at the effect of cooling velocity or cryopreservatives upon the survival or transplantation efficiency of frozen bone marrow cells or red blood cells (Lovelock, J. E. and Bishop, M. W. H., 1959, Nature 183:1394-1395; Ashwood-Smith, M. J., 1961, Nature 190:1204-1205; RoWe, A. W. and Rinfret, A. P., 1962, Blood 20:636; Rowe, A. W. and Fellig, J., 1962, Fed. Proc. 21:157; Rowe, A. W., 1966, Cryobiology 3(1):12-18; Lewis, J. P., et al., 1967, Transfusion 7(1):17-32; Rapatz, G., et al., 1968, Cryobiology 5(1):18-25; Mazur, P., 1970, Science 168:939-949; Mazur, P., 1977, Cryobiology 14:251-272; Rowe, A. W. and Lenny, L. L., 1983, Cryobiology 20:717; Stiff, P. J., et al., 1983, Cryobiology 20:17-24; Gorin, N. C., 1986, Clinics in Haematology 15(1):19-48).

The successful recovery of human bone marrow cells after long-term storage in liquid nitrogen has been described (1983, American Type Culture Collection, Quarterly Newsletter 3(4):1). In addition, stem cells in bone marrow were shown capable of withstanding cryopreservation and thawing without significant cell death, as demonstrated by the ability to form equal numbers of mixed myeloid-erythroid colonies in vitro both before and after freezing (Fabian, I., et al., 1982, Exp. Hematol 10:119-122). The cryopreservation and thawing of human fetal liver cells (Zuckerman, A. J., et al., 1968, J. Clin. Pathol. (London) 21(1):109-110), fetal myocardial cells (Robinson, D. M. and Simpson, J. F., 1971, In Vitro 6(5):378), neonatal rat heart cells (Alink, G. M., et al., 1976, Cryobiology 13:295-304), and fetal rat pancreases (Kemp, J. A., et al., 1978, Transplantation 26:260-264) have also been reported.

The injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-Sorbitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, Ilbery, P. L. T., ed., Butterworth, London, p. 59). In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4° C. until freezing, since DMSO concentrations of about 1% are toxic at temperatures above 4° C.

A controlled slow cooling rate is critical. Different cryoprotective agents (Rapatz, G., et al., 1968, Cryobiology 5(1):18-25) and different cell types have different optimal cooling rates (see e.g., Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1° to 3° C./minute from 0° C. to −80° C. In one embodiment, this cooling rate can be used for the amniotic fluid derived HSCs of the invention described herein. The container holding the cells must be stable at cryogenic temperatures and allow for rapid heat transfer for effective control of both freezing and thawing. Sealed plastic vials (e.g., Nunc, Wheaton CRYULES®) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, cells can be rapidly transferred to a long-term cryogenic storage vessel. In a preferred embodiment, the expanded HSCs can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum

In a particular embodiment, the cryopreservation procedure described in Current Protocols in Stem Cell Biology, 2007, (Mick Bhatia, et. al., ed., John Wiley and Sons, Inc.) is used and is hereby incorporated by reference. Mainly when the HSCs on a 10-cm tissue culture plate have reached approximately 50% confluency, the media within the plate is aspirated and the HSC s are rinsed with phosphate buffered saline. The adherent HSC are then detached by 3 ml of 0.025% trypsin/0.04% EDTA treatment. The trypsin/EDTA is neutralized by 7 ml of media and the detached HSC are collected by centrifugation at 200×g for 2 min. The supernatant is aspirated off and the pellet of HSCs is resuspended in 1.5 ml of media. An aliquot of 1 ml of 100% DMSO is added to the suspension of HSCs and gently mixed. Then 1 ml aliquots of this suspension of HSCs in DMSO are dispensed into CRYULES® in preparation for cryopreservation. The sterilized storage CRYULES® preferably have their caps threaded inside, allowing easy handling without contamination. Suitable racking systems are commercially available and can be used for cataloguing, storage, and retrieval of individual specimens.

Considerations and procedures for the manipulation, cryopreservation, and long-term storage of HSCs, particularly from bone marrow or peripheral blood can be found, for example, in the following references, incorporated by reference herein: Gorin, N.C., 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-minor techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, and 4,559,298 and all of these are incorporated hereby reference in their entirety.

Recovering Hematopoeitic Stem Cells from the Frozen State

Frozen HSCs are preferably thawed quickly (e.g., in a water bath maintained at 37°-41° C.) and chilled on ice immediately upon thawing. In particular, the cryogenic vial containing the frozen HSCs can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In a particular embodiment, the thawing procedure after cryopreservation is described in Current Protocols in Stem Cell Biology 2007 (Mick Bhatia, et. al., ed., John Wiley and Sons, Inc.) and is hereby incorporated by reference. Immediately after removing the cryogenic vial from the cryo-freezer, the vial is rolled between the hands for 10 to 30 sec until the outside of the vial is frost free. The vial is then held upright in a 37° C. water-bath until the contents are visibly thawed. The vial is immersed in 95% ethanol or sprayed with 70% ethanol to kill microorganisms from the water-bath and air dry in a sterile hood. The contents of the vial are then transferred to a 10-cm sterile culture containing 9 ml of media using sterile techniques. The HSCs can then be cultured and further expanded in a incubator at 37° C. with 5% humidified CO₂.

It may be desirable to treat the HSCs in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed HSCs. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

In a preferred, but not required, aspect of the invention, thawed cells are tested by standard assays of viability (e.g., trypan blue exclusion) and of microbial sterility as described herein, and tested to confirm and/or determine their identity relative to the recipient. In one embodiment, the viability of a thawed composition enriched in human amniotic fluid derived HSCs is at least 20% to at least 99%, and including all the percentages between 20-99%.

Methods for identity testing which can be used include but are not limited to HLA (the major histocompatibility complex in man) typing (Bodmer, W., 1973, in Manual of Tissue Typing Techniques, Ray, J. G., et al., eds., DHEW Publication No. (NIH) 74-545, pp. 24-27), and DNA fingerprinting, which can be used to establish the genetic identity of the cells. DNA fingerprinting (Jeffreys, A. J., et al., 1985, Nature 314:67-73) exploits the extensive restriction fragment length polymorphism associated with hypervariable minisatellite regions of human DNA, to enable identification of the origin of a DNA sample, specific to each individual (Jeffreys, A. J., et al., 1985, Nature 316:76; Gill, P., et al., 1985, Nature 318:577; Vassart, G., et al., 1987, Science 235:683), and is thus preferred for use.

### Formulation and Application

In one embodiment, a nucleic acid sequence, a vector carrying the nucleic acid sequence or an agent described in the methods herein administered to a subject or isolated HSCs comprise a non-cationic lipid for cytoplasmic and/or nuclear delivery, wherein the nucleic acid, vector or agent is stable and is used in biological extracellular fluids typically found in animals, particularly blood serum.

Liposomes, spherical, self-enclosed vesicles composed of amphipathic lipids, have been widely studied and are employed as vehicles of material transfer for in vivo administration of therapeutic agents. In particular, the so-called long circulating liposomes formulations which avoid uptake by the organs of the mononuclear phagocyte system, primarily the liver and spleen, have found commercial applicability. Such long-circulating liposomes include a surface coat of flexible water soluble polymer chains, which act to prevent interaction between the liposome and the plasma components which play a role in liposome uptake. Alternatively, hyaluronan has been used as a surface coating to maintain long circulation.

In one embodiment, the liposomes encapsulate the nucleic acid sequences, vectors, agents or even the viral particles. In one embodiment, the nucleic acid sequences, vectors or agents are condensed with a cationic polymer, e.g., PEI, polyamine spermidine, and spermine, or a cationic peptide, e.g., protamine and poly-lysine, and encapsulated in the lipid particle. The liposomes can comprise multiple layers assembled in a step-wise fashion.

Lipid materials are well known and routinely utilized in the art to produce liposomes. Lipids may include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

In addition, in order to prevent the uptake of the liposomes into the cellular endothelial systems and enhance the uptake of the liposomes into the tissue of interest, the outer surface of the liposomes may be modified with a long-circulating agent. The modification of the liposomes with a hydrophilic polymer as the long-circulating agent is known to enable to prolong the half-life of the liposomes in the blood

Liposomes encapsulating the nucleic acid sequences described herein can be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467).

The use of a therapeutically effective amount of the nucleic acid sequences, vectors or agents disclosed herein for expanding HSC production in a subject in need thereof should preferably include but is not limited to a composition of the nucleic acid sequences, vectors or agent in lactated Ringer's solution, and the composition is sterile. Lactated Ringer's solution is a solution that is isotonic with blood and intended for intravenous administration. Included are antioxidants, buffers, antibiotics and solutes that render the pharmaceutical compositions substantially isotonic with the blood of an intended recipient. In another embodiment, the composition comprises gene delivery vectors described herein. In another embodiment, the composition also includes water, polyols, glycerine and vegetable oils, and nutrients for cells, for example. Compositions adapted for parenteral administration can be presented in unit-dose or multi-dose containers, in a pharmaceutically acceptable dosage form. Such dosage forms, along with methods for their preparation, are known in the pharmaceutical and cosmetic art. Harry's Cosmeticology (Chemical Publishing, 7th ed. 1982); Remington's Pharmaceutical Sciences (Mack Publishing Co., 18th ed. 1990).

In one embodiment, dosage forms include pharmaceutically acceptable carriers that are inherently nontoxic and nontherapeutic in the amounts used. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In one embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like.

In one embodiment, other ingredients can be added, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In some embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocamne to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylaminoethanol, histidine, procaine, to name a few.

Pharmaceutical compositions can be administered by any known route. By way of example, the composition can be administered by a mucosal, pulmonary, topical, or other localized or systemic route (e.g., enteral and parenteral). The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

Administration can be systemic or local. In addition, it can be desirable to introduce the pharmaceutical compositions of the invention into the central nervous system by any suitable route, including intraventricular and intrathecal injection; intraventricular injection can be facilitated by an intraventricular catheter, for example, attached to a reservoir, such as an Omcana reservoir. Pulmonary administration can also be employed, e.g., by use of an inhaler or nebulizer, and formulation with an aerosolizing agent.

In one embodiment, the pharmaceutical formulation to be used for therapeutic administration must be sterile. Sterility is readily accomplished by filtration through sterile filtration membranes (e.g., 0.2 micron membranes). The pH of the pharmaceutical formulation typically should be about from 6 to 8.

In one embodiment, the composition can be delivered in a controlled release system. In one embodiment, a pump can be used (see Langer, supra; Sefton, CRC Crit. Ref. Biomed. Eng., 14:201 (1987); Buchwald et al., Surgery, 88:507 (1980); Saudek et al., N. Engl. J. Med., 321:574 (1989)). In another embodiment, polymeric materials can be used (see, Medical Applications of Controlled Release, Langer and Wise, eds. (CRC Press, Boca Raton, Fla. 1974); Controlled Drug Bioavailability, Drug Product Design and Performance, Smolen and Ball, eds. (Wiley, N.Y. 1984); Ranger and Peppas, Macromol. Sci. Rev. Macromol. Chem., 23:61 (1983); see also Levy et al., Science, 228:190 (1985); During et al., Ann. Neurol., 25:35 1 (1989); Howard et al., J. Neurosurg., 7 1:105 (1989)). Other controlled release systems are discussed in the review by Langer (Science, 249:1527-1533 (1990)). For examples of sustained release compositions, see U.S. Pat. No. 3,773,919, EP 58,481A, U.S. Pat. No. 3,887,699, EP 158,277A, Canadian Patent No. 1176565, U. Sidman et al., Biopolymers 22:547 (1983) and R. Langer et al., Chem. Tech. 12:98 (1982).

The therapeutically effective amounts to be administered will depend on the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art; however, that a lower dose or tolerable dose may be administered for medical reasons, psychological reasons or for virtually any other reason.

The dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. For gene therapy, viral vector should be in the range of 1×10⁶ to 10¹⁴ viral vector particles per application per patient.

In addition, in vitro or in vivo assays can optionally be employed to help identify optimal dosage ranges. The precise dose to be employed will also depend on the route of administration, and the seriousness of the condition being treated and should be decided according to the judgment of the practitioner and each subject's circumstances in view of, e.g., published clinical studies. Suitable effective dosage amounts, however, range from about 10 micrograms to about 5 grams about every 4 hour, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered.

### Peptides, Variants, and Peptidomimetics

Peptides and peptidomimetics can be chemically synthesized and purified by biochemical methods that are well known in the art such as solid phase peptide synthesis using t-Boc (tert-butyloxycarbonyl) or FMOC (9-flourenylmethloxycarbonyl) protection group described in “Peptide synthesis and applications” in Methods in molecular biology Vol. 298, Ed. by John Howl and “Chemistry of Peptide Synthesis” by N. Leo Benoiton, 2005, CRC Press, (ISBN-13: 978-1574444544) and “Chemical Approaches to the Synthesis of Peptides and Proteins” by P. Lloyd-Williams, et. al., 1997, CRC-Press, (ISBN-13: 978-0849391422). Solid phase peptide synthesis, developed by R. B. Merrifield, 1963, J. Am. Chem. Soc. 85 (14): 2149-2154, was a major breakthrough allowing for the chemical synthesis of peptides and small proteins. An insoluble polymer support (resin) is used to anchor the peptide chain as each additional alpha-amino acid is attached. This polymer support is constructed of 20-50 μm diameter particles which are chemically inert to the reagents and solvents used in solid phase peptide synthesis. These particles swell extensively in solvents, which makes the linker arms more accessible.

Organic linkers attached to the polymer support activate the resin sites and strengthen the bond between the amino acid and the polymer support. Chloromethyl linkers, which were developed first, have been found to be unsatisfactory for longer peptides due to a decrease in step yields. The PAM (phenylacetamidomethyl) resin, because of the electron withdrawing power of the acid amide group on the phenylene ring, provides a much more stable bond than the classical resin. Another alternative resin for peptides under typical peptide synthesis conditions is the Wang resin. This resin is generally used with the FMOC labile protecting group.

A labile group protects the alpha-amino group of the amino acid. This group should be easily removed after each coupling reaction so that the next alpha-amino protected amino acid may be added. Typical labile protecting groups include t-Boc and FMOC t-Boc is a very satisfactory labile group which is stable at room temperature and easily removed with dilute solutions of trifluoroacetic acid (TFA) and dichloromethane. FMOC is a base labile protecting group which is easily removed by concentrated solutions of amines (usually 20-55% piperidine in N-methylpyrrolidone). When using FMOC alpha-amino acids, an acid labile (or base stable) resin, such as an ether resin, is desired.

The stable blocking group protects the reactive functional group of an amino acid and prevents formation of complicated secondary chains. This blocking group must remain attached throughout the synthesis and may be removed after completion of synthesis. When choosing a stable blocking group, the labile protecting group and the cleavage procedure to be used should be considered.

After generation of the resin bound synthetic peptide, the stable blocknig groups are removed and the peptide is cleaved from the resin to produce a “free” peptide. In general, the stable blocking groups and organic linkers are labile to strong acids such as TFA. After the peptide is cleaved from the resin, the resin is washed away and the peptide is extracted with ether to remove unwanted materials such as the scavengers used in the cleavage reaction. The peptide is then frozen and lyophilized to produce the solid peptide. This is then characterized by HPLC and MALDI before being used. In addition, the peptide should be purified by HPLC to higher purity before use.

Commercial peptide synthesizing machines are available for solid phase peptide synthesis. For example, the Advanced Chemtech Model 396 Multiple Peptide Synthesizer and an Applied Biosystems Model 432A Peptide synthesizer. There are commercial companies that make custom synthetic peptide to order, e.g. Abbiotec, Abgent, AnaSpec Global Peptide Services, LLC. Invitrogen and rPeptide, LLC.

### Designing Peptide Mimetics

Methods of designing peptide mimetics and screening of functional peptide mimetics are well known in the art. One basic method of designing a molecule which mimics a known protein or peptide first identifies the active region(s) of the known protein (for example in the case of an antibody-antigen interaction one identifies which region(s) of the antibody enable binding to the antigen), and then searches for a mimetic which emulates the active region. Since the active region of the known protein is relatively small, it is hoped that a mimetic will be found which is much smaller (e.g. in molecular weight) than the protein, and correspondingly easier and cheaper to synthesis. Such a mimetic could be used as a convenient substitute for the protein, as an agent for interacting with the target molecule.

For example, Reineke et al. (1999, Nature Biotechnology, 17;271-275) disclose a mimic molecule which is designed to mimic a binding site of the interleukin-10 protein, where the mimic molecule was designed using a large library of synthesized short peptides, each of which corresponded to a short section of interleukin 10. The binding of each of these peptides to the target (in this case an antibody against interleukin-10) was then tested individually by an assay technique, to identify potentially relevant peptides. Phage display libraries of peptides and alanine scanning method can be used.

Other methods for designing peptide mimetic to a particular peptide or protein include The Chemical Computing Group's Molecular Operating Environment” (M.O.E.) software, European Patent EP1206494, the SuperMimic program by Andrean Goede et. al. 2006 BMC Bioinformatics, 7:11; and MIMETIC program by W. Campbell et al., 2002, Microbiology and Immunology 46:211-215. The SuperMimic program is designed to identify compounds that mimic parts of a protein, or positions in proteins that are suitable for inserting mimetics. The application provides libraries that contain peptidomimetic building blocks on the one hand and protein structures on the other. The search for promising peptidomimetic linkers for a given peptide is based on the superposition of the peptide with several conformers of the mimetic. New synthetic elements or proteins can be imported and used for searching. The MIMETIC computer program, which generates a series of peptides for interaction with a target peptide sequence, is taught by W.Campbell et. al., 2002. In depth discussion of the topic is reviewed in “Peptide Mimetic Design with the Aid of Computational Chemistry” by James R. Damewood Jr. in Reviews in Computational Chemistry Reviews in Computational Chemistry, Jan 2007, Volume 9 Book Series: Reviews in Computational Chemistry, Editor(s): Kenny B. Lipkowitz, Donald B. BoydPrint ISBN: 9780471186397 ISBN: 9780470125861 Published by John Wiley &Sons, Inc.; and in T. Tselios, et. al., Amino Acids, 14: 333-341, 1998.

Methods for preparing libraries containing diverse populations of peptides, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). One skilled in the art understands that a peptide can be produced in vitro directly or can be expressed from a nucleic acid, which can be produced in vitro. Methods of synthetic peptide and nucleic acid chemistry are well known in the art.

A library of peptide molecules also can be produced, for example, by constructing a cDNA expression library from mRNA collected from a tissue of interest. Methods for producing such libraries are well known in the art (see, for example, Sambrook et al., Molecular Cloning: A laboratory manual (Cold Spring Harbor Laboratory Press 1989), which is incorporated herein by reference). Preferably, a peptide encoded by the cDNA is expressed on the surface of a cell or a virus containing the cDNA.

The invention can be defined by any of the following alphabetized paragraphs:
[A] A method of expanding ex vivo a population of hematopoietic stem cells (HSCs), the method comprising contacting an isolated HSC with a nucleic acid sequence comprising miR-125a, thereby expanding ex vivo a population of HSCs.
[B] The method of paragraph A, wherein the HSC is isolated from peripheral blood, umbilical cord blood, or bone marrow from a subject.
[C] The method of paragraph A, wherein the nucleic acid sequence comprises SEQ. ID. No. 1 or 2.
[D] The method of paragraph A, wherein the nucleic acid sequence comprising miR-125a is expressed from a vector containing a nucleic acid sequence encoding miR-125a or a precursor thereof.
[E] The method of paragraph D, wherein the vector is selected from a plasmid, cosmid, phagemid, or virus.
[F] The method of paragraph 4, wherein the vector further comprises one or more expression elements operatively linked to the nucleic acid sequence encoding miR-125a or a precursor thereof for expression in HSCs.
[G] The method of paragraph F, wherein the expression element comprises a promoter or enhancer and combinations thereof.
[H] The method of any of paragraphs A-G, further comprising expanding the HSCs for at least one cell doubling ex vivo.
[I] The method of paragraph H, further comprising cryopreserving the expanded HSCs.
[J] The method of paragraph H or I, wherein the expanded HSCs are used therapeutically in a subject.
[K] The method of paragraph J, wherein the expanded HSCs are administered intravenously.
[L] A method of expanding ex vivo a production of HSC, the method comprising contacting an isolated HSC with a therapeutically effective amount of an agent that increases the expression miR-125a in the cell.
[M] The method of paragraph L, wherein the agent is selected from a group consisting of a small molecule, nucleic acid, nucleic acid analogue, protein, antibody, peptide, aptamer and variants or fragments thereof.
[N] The method of paragraph L, wherein the therapeutically effective amount of an agent is provided by administering a pharmaceutical composition comprising the agent and a pharmaceutically acceptable carrier.
[O] A method of inhibiting HSC self-renewal in a subject in need thereof, the method comprises administering a therapeutically effective amount of an agent that inhibits mir-125a to a subject.
[P] A method of treating leukemia in a subject in need thereof, the method comprising administering a therapeutically effective amount of an agent that inhibits mir-125a in a cell to a subject.
[Q] The method of paragraph O or P, wherein the agent is selected from the group consisting of an antagomir of mir-125a, an anti-mir-125a oligonucleotide, an antisense oligonucleotide to mir-125a, an siRNA to mir-125a, and a locked nucleic acid that anneals to mir-125a.
[R] Use of a nucleic acid sequence comprising miR-125a for expanding a production of HSCs in a subject in need thereof.
[S] Use of a nucleic acid sequence comprising miR-125a for the manufacture of medicament for expanding a production of HSCs in a subject in need thereof.
[T] The use of paragraph R or S, wherein the nucleic acid sequence comprising miR-125a comprises SEQ. ID. No. 1 or 2.
[U] The use of paragraph R or S, wherein the nucleic acid sequence comprising miR-125a is expressed from a vector containing a nucleic acid sequence encoding miR-125a or a precursor thereof.
[V] The use of paragraph U, wherein the vector is selected from a plasmid, cosmid, phagemid, or virus.
[W] The use of paragraph V, wherein the vector further comprises one or more expression elements operatively linked to the nucleic acid sequence encoding miR-125a or a precursor thereof for expression in HSCs.
[X] The use of paragraph W, wherein the expression element comprises a promoter or enhancer and combinations thereof.
[Y] The use of paragraph R or S, wherein the subject is a mammal.
[Z] The use of paragraph Y, wherein the mammal is a human.
[AA] The use of paragraph R or S, wherein the subject has received, will receive or is concurrently receiving chemotherapy or radiation therapy.
[BB] The use of paragraph AA, wherein the subject has a disorder selected from the group consisting of myeloma, non-Hodgkin's lymphoma, Hodgkins lyphoma and leukaemia.
[CC] The use of paragraph BB, wherein the subject has received, will receive or is concurrently receiving granulocyte colony-stimulating factor (G-CSF).
[DD] The use of paragraph R or S, wherein the subject has a disorder characterized by a lack of functional blood cells.
[EE] The use of paragraph DD, wherein the disorder is a platelet deficiency.
[FF] The use of paragraph EE, wherein the disorder is an anemia.
[GG] The use of paragraph FF, wherein the anemia is selected from the group consisting of aplastic anemia, sickle cell anemia, fanconi's anemia and acute lymphocytic anemia.
[HH] The use of paragraph DD, wherein the disorder is a neutropenia.
[II] The use of paragraph R or S, wherein the subject has a disorder characterized by a lack of functional immune cells.
[JJ] The use of paragraph II, wherein the immune cells are T or B lymphocytes.
[KK] The use of paragraph II, wherein the disorder is selected from the group consisting of lymphocytopenia, lymphorrhea, lymphostasis and AIDS.
[LL] The use of paragraph R or S, wherein the subject has received, will receive or is receiving an immuno-suppressive drug.
[MM] The use of paragraph R or S, wherein the subject is a stem cell donor.
[NN] The use of paragraph R or S, wherein administering is intravenous, intradermal, intramuscular, intraarterial, intralesional, percutaneous, subcutaneous, or by aerosol.
[OO] A method of expanding hematopoietic stem cell (HSC) production in a subject in need thereof, the method comprising providing a therapeutically effective amount of a nucleic acid sequence comprising miR-125a to the subject, thereby expanding HSC production in the subject.
[PP] The method of paragraph OO, wherein the nucleic acid sequence comprising miR-125a comprises SEQ. ID. No. 1 or 2.
[QQ] The method of paragraph PP, wherein the nucleic acid sequence comprising miR-125a is expressed from a vector containing a nucleic acid sequence encoding miR-125a or a precursor thereof.
[RR] The method of paragraph OO, wherein the therapeutically effective amount of a nucleic acid sequence comprising miR-125a is provided by administering a pharmaceutical composition comprising i) a nucleic acid sequence comprising miR-125a or ii) a vector expressing a nucleic acid sequence encoding miR-125a or a precursor thereof.
[SS] The method of paragraph QQ or RR, wherein the vector is selected from a plasmid, cosmid, phagemid, or virus.
[TT] The method of paragraph SS, wherein the vector further comprises one or more expression elements operatively linked to the nucleic acid sequence encoding miR-125a or a precursor thereof for expression in HSCs.
[UU] The method of paragraph TT, wherein the expression element comprises a promoter or enhancer and combinations thereof.
[VV] The method of paragraph OO, wherein the subject is a mammal.
[WW] The method of paragraph VV, wherein the mammal is a human.
[XX] The method of paragraph OO, wherein the subject has received, will receive or is concurrently receiving chemotherapy or radiation therapy.
[YY] The method of paragraph XX, wherein the subject has a disorder selected from the group consisting of myeloma, non-Hodgkin's lymphoma, Hodgkins lyphoma and leukaemia.
[ZZ] The method of paragraph YY, wherein the subject has received, will receive or is concurrently receiving granulocyte colony-stimulating factor (G-CSF).
[AAA] The method of paragraph OO, wherein the subject has a disorder characterized by a lack of functional blood cells.
[BBB] The method of paragraph YY, wherein the disorder is a platelet deficiency.
[CCC] The method of paragraph YY, wherein the disorder is an anemia.
[DDD] The method of paragraph CCC, wherein the anemia is selected from the group consisting of aplastic anemia, sickle cell anemia, fanconi's anemia and acute lymphocytic anemia.
[EEE] The method of paragraph DDD, wherein the disorder is a neutropenia.
[FFF] The method of paragraph OO, wherein the subject has a disorder characterized by a lack of functional immune cells.
[GGG] The method of paragraph FFF, wherein the immune cells are T or B lymphocytes.
[HHH]The method of paragraph FFF or GGG, wherein the disorder is selected from the group consisting of lymphocytopenia, lymphorrhea, lymphostasis and AIDS.
[III] The method of paragraph OO, wherein the subject has received, will receive or is receiving an immuno-suppressive drug.
[JJJ] The method of paragraph OO, wherein the subject is a stem cell donor.
[KKK] The method of paragraph OO, wherein administering is intravenous, intradermal, intramuscular, intraarterial, intralesional, percutaneous, subcutaneous, or by aerosol.
[LLL] A method of expanding HSC production in a subject in need thereof, the method comprising providing a therapeutically effective amount of an agent that increases the expression miR-125a to the subject, thereby expanding HSC production in the subject.
[MMM] The method of paragraph LLL, wherein the agent is selected from a group consisting of a small molecule, nucleic acid, nucleic acid analogue, protein, antibody, peptide, aptamer and variants or fragments thereof.
[NNN] The method of paragraph LLL, wherein the therapeutically effective amount of an agent is provided by administering a pharmaceutical composition comprising the agent and a pharmaceutically acceptable carrier.
[OOO] The method of paragraph J, wherein the HSC are administered to a subject in a pharmaceutical composition comprising the HSC and a pharmaceutically acceptable carrier.
[PPP] The method of paragraph O, wherein the therapeutically effective amount of an agent that inhibits miR-125a is provided by administering to a subject in a pharmaceutical composition comprising the agent that inhibits miR-125a and a pharmaceutically acceptable carrier.
[QQQ] The method of paragraph R or S, wherein the nucleic acid sequence comprising miR-125a is administered to a subject in a pharmaceutical composition comprising nucleic acid sequence comprising miR-125a and a pharmaceutically acceptable carrier.
[RRR] A method of inhibiting HSC self-renewal ex vivo, the method comprises contacting the HSC with an agent that inhibits miR-125a.

This invention is further illustrated by the following examples which should not be construed as limiting.

The contents of all references cited throughout this application, as well as the figures and table are incorporated herein by reference.

### EXAMPLES

### Materials and Methods

### Mice and Tissue Processing

The Subcommittee on Research Animal Care of the Massachusetts General Hospital approved all animal work according to federal and institutional policies and regulations. The Dicerlox/lox mice were described in Cobb, B. S., et al., 2006, J Exp Med 203:2519-2527. Tail DNA was used for genotyping by PCR following procedures described (Cobb, B. S., et al., 2006, supra; Hartner, J. C., et al., 2009, Nature immunology 10:109-115).

All other mice including C57B16/J, B6.SJL-Ptprca Pep3b/BoyJ and MxCre mice were purchased from the Jackson Laboratory. Adult mice of 8-12 weeks of age were used. One full course of pIpC was administered by 7 consecutive i.p. doses given every other day (25 mg/kg) (Sigma). 9 Gy of γ-irradiation was delivered to all transplantation recipient mice 3-16 hours before transplantation via lateral tail vein injection. Peripheral blood samples were obtained by lateral tail vein bleeding and were collected into EDTA coated blood collection tubes (BD). Complete blood counts were measured using a HEMAVET 850FS.

### MicroRNA Expression Constructs

MicroRNA expression constructs were cloned into pMIRWAY-GFP, a MSCV-based retrovirus vector which expresses GFP under a PGK promoter, which were used for microRNA expression before in vitro and in vivo (Lu, J., et al., 2008, Dev Cell 14:843-853). Expression fragments for mmu-miR-99b-let-7e-miR-125a-cluster and mmu-miR-125a were PCR amplified from C57BL6/J mouse genomic DNA, which were prepared using the QIAGEN® DNeasy Kit. The core primers used for amplifying the murine miR-99b-let-7e-miR-125a-cluster are: 5′-GAAGTCAGGTCTCTAACCAG-3′ (SEQ. ID. No. 4) and 5′-CTTCAAGCTCATTTCTGCACAG-3′ (SEQ. ID. No. 5); the core primers for amplifying mmu-miR-125a are 5′-CCAAGAGTTCTTGATAGGAG-3′ (SEQ. ID. No. 6) and 5′-CTTCAAGCTCATTTCTGCACAG-3′ (SEQ. ID. No. 7). Viral production was performed in 293-T cells following described procedures (Lu, J., et al., 2008, supra).

### Bone Marrow Transplantation

For 1:1 competitive transplantation, 5×10⁵ test (CD45.2+) whole bone marrow cells were mixed with 5×10⁵ CD45.1+ competitor cells and injected into lethally irradiated (9 Gy) recipient B6-SJL (CD45.1+) mice. Engraftment was analyzed by CD45.2+ donor contribution using FACS analysis at multiple time points following transplantation. pIpC injections were initiated at least 5 weeks post transplantation.

Viral transduction of the bone marrow was as described previously (Lu, J., et al., 2008, supra). Alternatively, the same culture condition was used with FACS-purified hematopoietic stem/progenitors cells. Each recipient mouse received 1,000 LKS or 10,000 progenitors together with 2.5×10⁵ fresh whole bone marrow cells as support.

For limiting dilution assays, bone marrow from primary transplantation recipients was harvested 4 to 5 months post transplantation and sorted based on GFP. 2×10⁶, 2×10⁵, 5×10⁴ and 1×10⁴ GFP+ cells were mixed with 2×10⁵ wild type bone marrow and injected into lethally irradiated recipients. Engraftment was monitored by GFP+ cells using FACS analysis. The frequencies of competitive repopulating units were calculated using the L-Calc software. Greater than or equal to 1% GFP+ cells in all three lineages (Mac-1+, B220+ and CD3+) was used to determine whether an animal had a positive engraftment.

### Flow Cytometry and Cell Sorting

Long-term HSCs, short-term HSCs and multipotent progenitors were sorted based on their expression of lineage markers as well as c-Kit, Sca-1, CD34 and Flk2 expression (LT HSCs: Lin-Kit+Sca+CD34-Flk2−, ST HSCs: Lin-Kit+Sca+CD34-Flk2+ and MPPs: Lin-Kit+Sca+CD34+Flk2+). Lineage staining used a cocktail of biotinylated anti-mouse antibodies to Mac-1a (CD11b), Gr-1(Ly-6G/C), Ter119 (Ly-76), CD3, CD4, CD8a (Ly-2), and B220 (CD45R; BD Biosciences). For detection and sorting we used streptavidin conjugated with PE/Cy7, c-Kit-APC (CD117), Flk2-PE (CD135), CD34-FITC (all from BD Biosciences), and Scal-PE/Cy5.5 (Ly 6A/E; Caltag Laboratories). For congenic strain discrimination, anti-CD45.1-PE and anti-CD45.2 FITC antibodies (BD Biosciences) were used. For the apoptosis assay 7-AAD and AnnexinV-APC (BD Biosciences) were used. For the intracellular detection of Caspase-3 and Ki67, bone marrow cells were fixed and permeabilized using BD Cytofix/Cytoperm Fixation/Permeabilization Solution Kit (BD Biosciences) according to the manufacturer's recommendations.

### MicroRNA Expression Profiling and Data Processing

MicroRNA expression profiling was performed following our previously published protocol (Lu, J., et al., 2008, supra). Long-term HSCs, short-term HSCs, multipotent progenitors, Lin-Kit+Sca+ cells, Lin-Kit+Sca− cells, Lin-Kit-Sca+ cells, Lin-cells and unfractionated whole bone marrow cells were prepared for total RNA using TriZol (Invitrogen) in replicates. For rare populations, cells from multiple mice were pooled. To perform microRNA profiling, 60 ng of total RNA were used for each sample. Data were normalized according to total RNA input, as described (Lu, J., et al., 2005, Nature 435:834-838). An alternative normalization methodology, assuming equal total microRNA content between samples, produces similar result on miR-99b, let-7e and miR-125a (data not shown).

### Methylcellulose Colony Formation

Bone marrow cells were plated into methylcellulose M3434 (StemCell Technologies) and grown for 10-14 days before scored and photographed. Isolated macroscopic colonies were manually plucked. These colonies were then digested in PCR buffers containing 0.4 ng/ml of proteinase K and 1% tween-20 for 1 hour at 56° C. followed by 15 minutes of heat inactivation at 94° C. The digestion mix was used directly for PCR reactions. Interferon-β was added at 1,000 IU/ml. Antagomirs (Dharmacon) were added at 50 μg/ml. Antagomir against miR-125a: 5′ mC(*)mA(*)mCmAmGmGmUmUmAmAmAmGmGmGmUmCmUmCmAmG(*)mG(*)mG (*)mA(*)(3′-Chl) (SEQ. ID. No. 8). Control antagomir: 5′ mC(*)mU (*)mCmG mCmGmU mAmGmA mAmGmA mGmUmA mGmGmU (*)mG(*) mG(*)mA (*) (3′-Chl) (SEQ. ID. No. 9). mN stands for 2′-OMe modification; (*) stands for phosphorothioate bond. 3′-Chl stands for 3′-cholestrol modification.

### Luciferase Reporter Assay

293T cells were plated in 96-well plates at 5000 cells per well the day before transfection. Transfection was carried out in triplicate using FuGENE 6 (Roche), with 150 ng of plasmid mixture (135 ng of expression vector and 15 ng of reporter vector in the psiCHECK2 backbone). Luciferase assays for both firefly and renilla luciferase were performed 2 days after transfection using the Dual-Glo Luciferase assay kit (Promega). Luminescence was quantitated using Luminoskan Ascent. Renilla luciferase readings were normalized against the firefly luciferase activity in the corresponding well.

### Data Analysis

Except as specified where representative data are shown, all data is presented as mean±SD. p values were calculated using 2 tailed, unequal variance Student t test.

### Example 1

### Dicer Deletion Induces Multi-Lineage Cytopenia in a Cell-Autonomous Manner

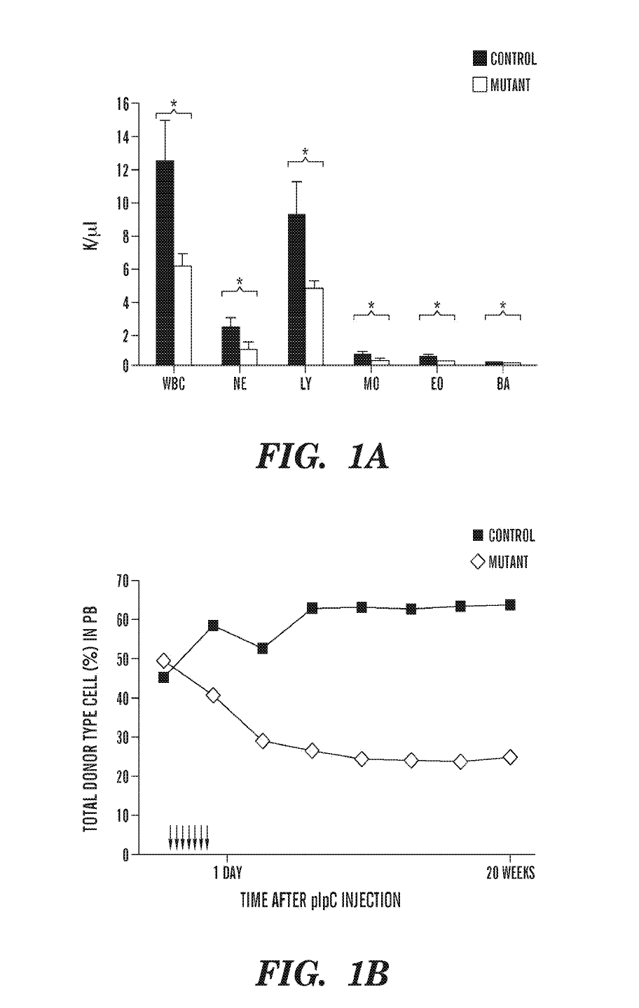

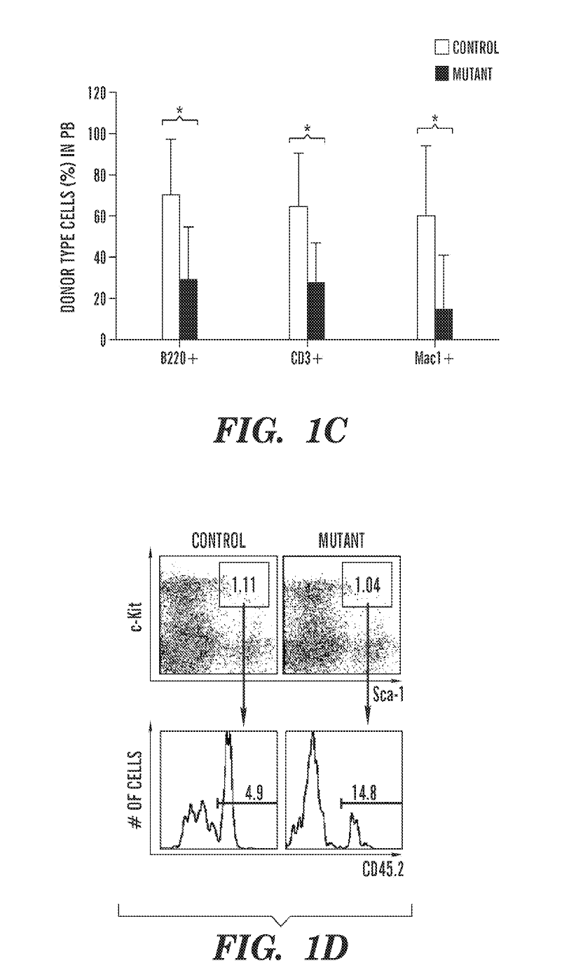

Dicer^{lox/lox} mice were bred with MxCre mice, which express Cre recombinase in response to interferons and can be experimentally induced with high efficiency via peritoneal injection of synthetic polyI:polyC (pIpC). Mice with the genotypes of Cre⁺Dicer^{lox/lox} (termed “mutant”) and Cre⁺Dicer^{wt/wt} or Cre⁺Dicer^{lox/wt} littermates (termed “control”) were chosen for experiments, as all experiments performed using the Cre⁺Dicer^{wt/wt} and Cre⁺Dicer^{lox/wt} showed indistinguishable results (data not shown). Mutant mice displayed extensive cytopenia in all nucleated lineages and platelets one day after the full dose of pIpC (FIG. 1A and Table 1), while no difference was observed in hematopoietic parameters prior to Cre activation (Table 2). The multi-lineage defect was only seen transiently and was completely resolved 3 weeks post pIpC (Table 3). All mice remained healthy for an additional >8 months (data not shown).

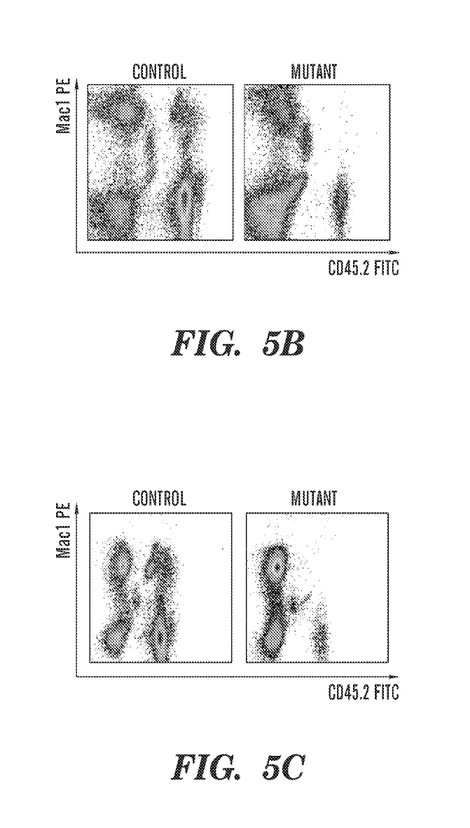

The multi-lineage cytopenia could be a result of impairment in either a common primitive multipotent population, or in multiple independent committed lineages (e.g. T and B cells). To assess whether HSCs affected by Dicer loss, whole bone marrow from control or mutant mice (CD45.2+) were mixed in equal numbers with competitor bone marrow (CD45.1+) and transplanted into lethally irradiated recipient mice (CD45.1+). The contribution to T cells, B cells and myeloid cells in the peripheral blood (PB) was monitored over time (FIGS. 1B and C, FIG. 5B). While both groups showed ˜50% donor type (CD45.2+) reconstitution in all lineages prior to pIpC injection, reconstitution from the mutants declined after pIpC treatment, and remained declined until 20 weeks post transplantation, when donor contribution is primarily coming from stem cells. The reduction in reconstitution could also be observed in secondary transplant recipients, underscoring the importance of Dicer in HSCs (FIG. 5C). However, the presence of T cells, B cells and myeloid cells were all readily detectable in roughly normal proportions within the donor cell compartment (CD45.2+) (FIG. 5A). The normal lineage distribution within the remaining donor type cells suggests that the impairment was within a common primitive multipotent population, rather than separate committed lineages, as it is unlikely that Dicer loss would concurrently and proportionately affect the lineages to the same extent. Furthermore, the donor cell contribution to immuno-phenotypic stem cells was similarly reduced (FIG. 1D), and this reduction was confirmed by functional assessment in secondary transplants (data not shown).

### Example 2

### HSCs with Incompletely Deleted Dicer are Responsible for the Hematopoietic Recovery

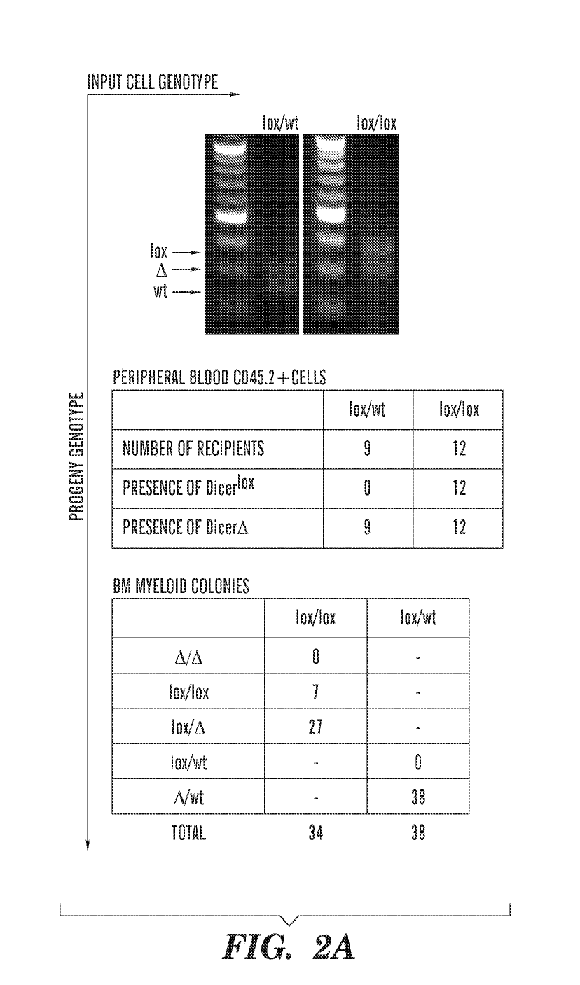

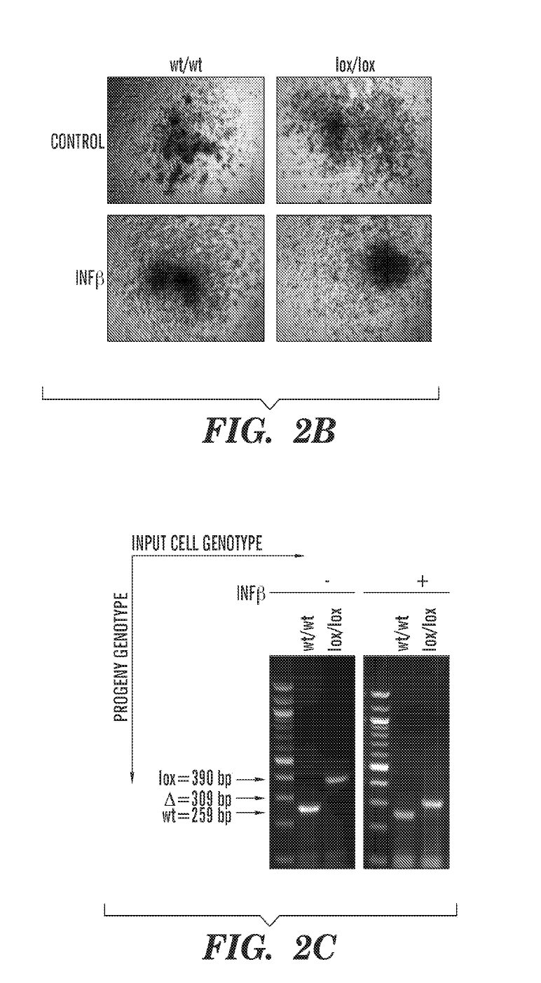

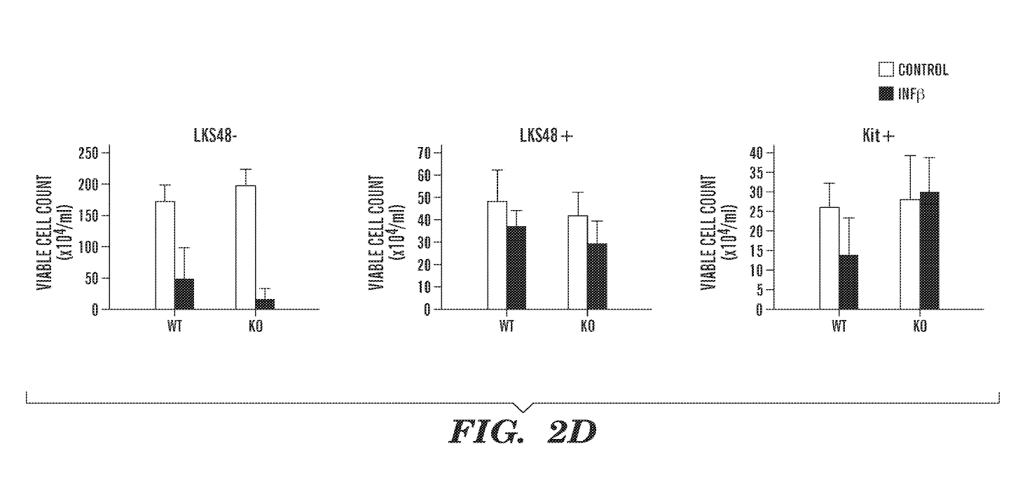

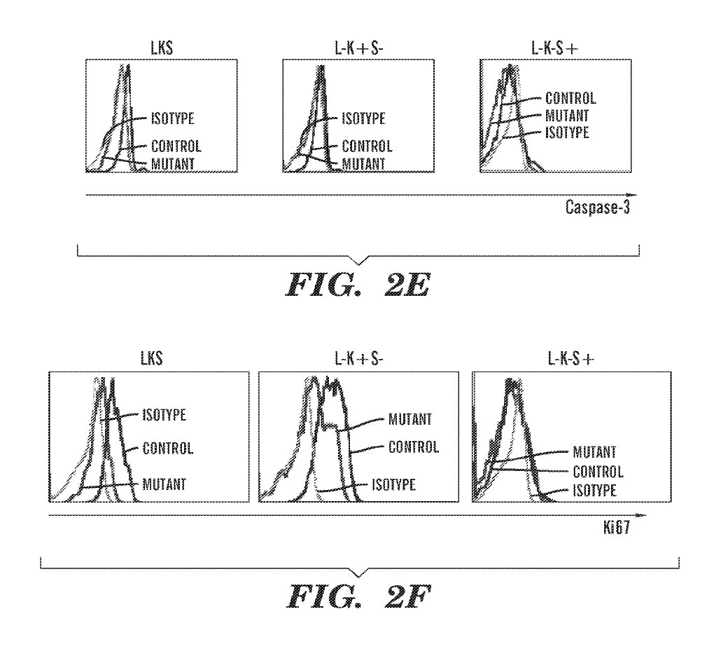

To reconcile the drastic reduction in overall donor cell reconstitution and an apparent absence of abnormality with the remnant donor hematopoiesis, the deletion status of the floxed Dicer allele was investigated. Donor type cells from the peripheral blood were FACS sorted 6 months post-transplant and genomic DNA was tested for Dicer deletion (FIG. 2A). All mice transplanted with control Cre⁺Dicer^{lox/wt} cells showed complete deletion of all the loxed alleles, attesting to the expected high efficiency of the MxCre system. However, mice received Cre⁺Dicer^{lox/wt} cells all harbored one “loxed” allele (functionally wild type allele) in addition to one deleted allele (Δ). To enable the assay at a clonal level, donor type bone marrow were FACS sorted and plated into methylcellulose for clonal amplification. While there was no difference in the types and numbers of colonies between the control and mutant cells (data not shown), no colony could be identified with a Dicer^{A/A} genotype (0/34) (FIG. 2A, bottom table). This is not due to the inefficiency of the MxCre system, as the control Dicer^{lox/wt} colonies all showed a Dicer^{Δ/wt} genotype (38/38). Dicer^{Δ/Δ} colonies were also completely absent from bone marrow cells of non transplanted animals received pIpC while all control colonies showed a Dicer^{Δ/wt} genotype from the Dicer^{lox/wt} cells (Table 5). These experiments demonstrate that the HSCs with incompletely deleted Dicer are responsible for the hematopoietic recovery and highlight the essentiality of Dicer in HSC maintenance.

### Example 3

### Differential Sensitivity of Primitive Hematopoietic Cells to Dicer Loss

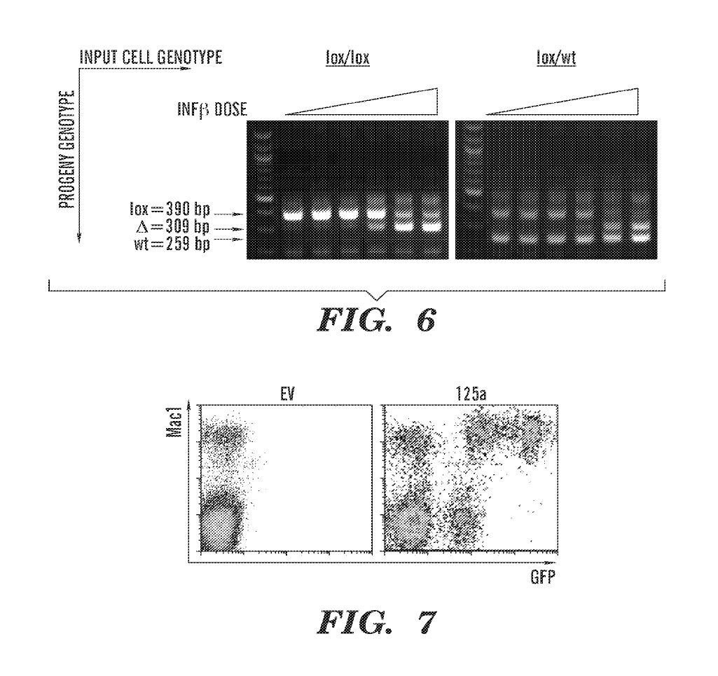

To directly test the sensitivity to Dicer loss among different primitive hematopoietic populations, LKS cells containing all the long term repopulating ability, and the Lin-c-Kit⁺Sca⁻ (LKS−) population containing all the non-lymphoid progenitors were FACS sorted and cultured in vitro in the presence of interferon β (INF β), the activator of Cre expression. Under conditions where Dicer is efficiently deleted (FIG. 6), Cre⁺Dicer^{lox/lox} LKS cells showed markedly increased death (7-AAD⁺/Anexin V⁺ cells) (data not shown). As a result, viable cell output from CD48-LKScells, CD48⁺ LKS and LKS⁻ was differentially affected by Dicer loss. While INF-β induced a cytostatic response in the control CD48-LKS cells, mutant CD48-LKS cells displayed further inhibition (˜30% of that of control) (FIG. 2D). In sharp contrast, the closely related CD48⁺ LKS population behaved similarly in both control and mutant cells, and the mutant LKS+ progenitors showed even somewhat superior cell output in this setting. Taken together, these experiments demonstrate that there is differential dependence on Dicer among the closely related early hematopoietic cells and the CD48-LKS cells display distinctive sensitivity to Dicer loss.

To directly test differentiation-stage-specific sensitivity to Dicer loss, several bone marrow populations were examined for apoptosis immediately following pIpC treatment. LKS cells (containing all long-term repopulating ability), Lin-Kit+Sca− (LK+S−) cells (containing myeloid progenitors), and another heterogeneous population Lin-Kit-Sca+ (LK-S+) were examined for caspase-3 activation. Mutant LKS cells displayed a marked increase in apoptosis, whereas the L-K+S− myeloid progenitor population and L-K−S+ population were not affected (FIG. 2E). Meanwhile, mutant bone marrow demonstrated increased Ki67 staining suggesting a compensatory response to cell loss possibly accounting for the transient nature of the drop in mature blood cell production (FIG. 2F).

If Dicer is dispensable for progenitors, which can form colonies in methylcellulose, colonies with a Dicer^{Δ/Δ} genotype should be preserved when competition is not present. Control and mutant bone marrow cells were plated into methylcellulose in the absence or presence of INF-β. Distinctive colonies were observed in the mutant colony cultures with INF-β (FIG. 2B) and subsequent PCR confirmed that these colonies indeed had fully deleted Dicer (FIG. 2C). Thus Dicer null status is not required for colony forming progenitor survival or growth. The absolute absence of Dicer^{Δ/Δ} colonies from bone marrow where Cre was induced in vivo may be attributable to the HSC defect precluding progenitor generation in fully deleted cells. Only HSCs that harbor incompletely deleted Dicer provided descendent colony forming progenitor cells.

### Example 4

### miR-99b-let-7e-miR-125a Cluster is Exclusively Expressed in Long-Term HSCs

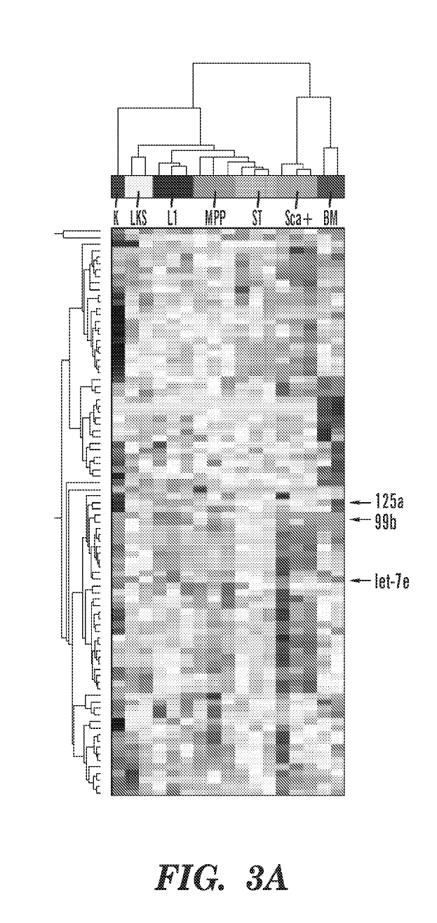

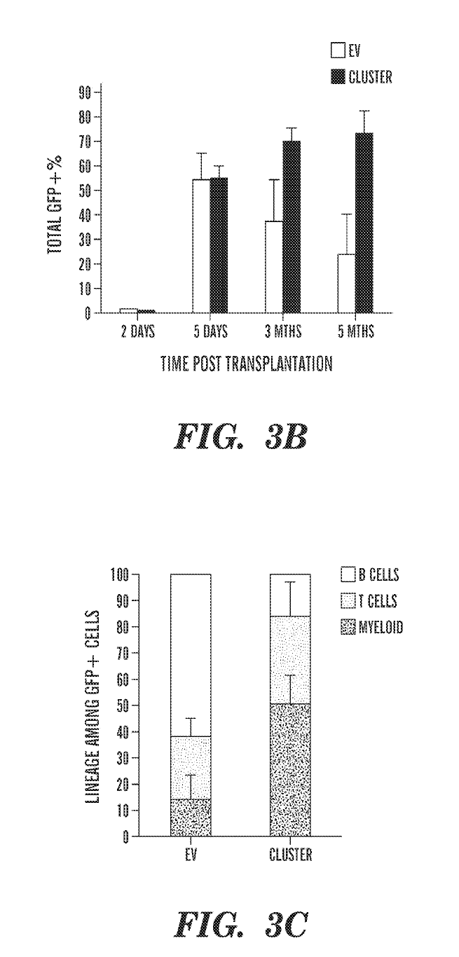

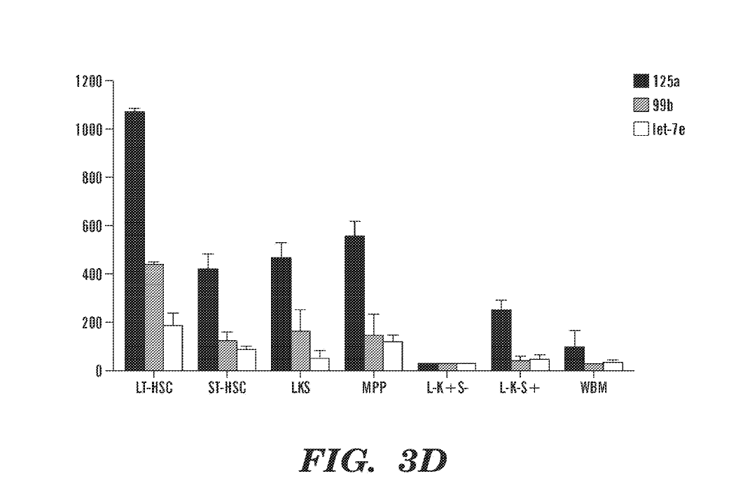

Dicer is involved in the maturation of both mature microRNAs and siRNAs with microRNAs better defined as demarcating developmental stage. The global microRNA expression were examined in the primitive hematopoietic compartments with varying degree of self-renewal ability (FIG. 3A). The expression of multiple microRNAs correlates with the ability to self-renew (Table 4), thus they may serve regulatory roles, or merely as markers, for the self renewal state. Of particular interest, three microRNAs, miR-99b, let-7e and miR-125a, are highly expressed in long-term (LT) HSCs compared to other populations (FIG. 3D). These three microRNAs scored high in LT-HSCs display a complete evolutionary conservation and organize in a cluster spanning a ˜1 kb region on chromosome 19 in human and in mice (data not shown). These features prompted the testing of the function of this microRNA cluster in regulating HSC self-renewal.

GFP-expressing bone marrow cells, when forced to express this cluster, showed enhanced reconstitution in all major blood lineages after long-term reconstitution, contrasting that of control animals whose GFP+ cells decline over time (FIGS. 3B and C). This increase in multi-lineage reconstitution was accentuated in secondary transplantion (data not shown), suggesting this microRNA cluster has positively modified the activity of HSCs.

### Example 5

### miR-125a Positively Regulates HSC Self-Renewal

Individual microRNAs in the cluster were analyzed for an impact on stem cell function. miR-125a alone, but not miR-99b and let-7e, provided increased long-term multi-lineage reconstitution (FIGS. 4A, B, D and E). As enhanced survival or proliferation of the committed progenitors can present a similar phenotype, purified common lymphoid progenitors (CLP), common myeloid progenitors (CMP), granulocyte and macrophage progenitors (GMP) and megakaryocyte and erythroid progenitors (MEP), as well as the LKS cells were tested for their ability to gain or maintain, partly or entirely, the stem cell property of self renewal after miR-125a transduction (FIG. 4F). The production of mature cells in the peripheral blood from the progenitor populations (CLP, CMP, GMP and MEP) was minimal and disappeared completely in the early weeks (3-6 weeks) post transplantation. LKS cells transduced with the control vector generated <2% of peripheral blood mononuclear cells. In contrast, LKS cells transduced with miR-125a reconstituted >70% of peripheral blood cells starting from 3 weeks post transplantation and lasting through 5 months (FIG. 4F) and secondary transplantation (data not shown). These results highly indicate that miR-125a positively modified the production capacity of HSCs and HSC number, but is not capable of delivering self-renewal ability to committed progenitors.

To determine whether miR-125a affects the quantity of reconstituting stem cells, competitive limiting dilution assay (LDA) analysis was performed using total GFP+ bone marrow cells from the primary transplants. The ability of LDA analysis to reliably reflect stem cell number is dependent upon the productivity of the test stem cells. Reconsitution by the transplanted test stem cells is scored based on a minimal representation of the test cell genotype in the blood of recipient animals. Therefore, in settings where differentiation and production of mature blood cells is altered within the stem cell population there can be skewing of the results. For example, if differentiation of mature cells was markedly impaired the stem cell number would be underestimated due to low scoring of donor genotype in the blood. Therefore, the ability of miR-125a transduced cells to produce mature cell types was examined (data not shown). Having documented that multi-lineage cells were not substantially affected, both myeloid and lymphoid lineages were scored in the LDA. It was estimated that one in every ˜5 million vector-transduced cells to be stem cells, while one in every ˜1 million of miR-125a-transduced cells to be such. Considering the overall >2 fold GFP⁺ percentage increase in the primary transplants, miR-125a has expanded HSC numbers by at least 10 fold. The increase in stem cell numbers can have a reduced dependence for a specialized microenvironment, or niche, since robust stem cell activity was preserved during an 18 day in vitro culture period if miR-125a was expressed (FIG. 4C and FIG. 7). Conversely, inhibiting the activity of miR-125a by an antagomir, obliterated the presence of high proliferative potential colonies and secondary colonies in methylcellulose assays (FIG. 4G).

In a subsequent LDA analysis, using >1% of both lineages as the criteria for reconstitution, it was estimated that one in every ˜2 million control-vector-transduced cells to be stem cells, whereas one in every ˜0.6 million of miR-125a-transduced cells to be such (FIG. 4H). Total GFP+ bone marrow cells were FACS-sorted from primary transplantation recipients 5 months post-transplant. Four different cell doses (2,000,000, 200,000, 50,000 and 10,000) were tested with 10 animals per cell dose for either control- or miR-125a-transduced cells. 250,000 un-fractionated wild-type whole bone marrow cells were used to provide competitors and radio-protection. A responder was called when myeloid (Mac1+) and lymphoid lineages (B220+ and CD3+) showed ≧1% GFP+ cells. Stem cell frequency was calculated using the L-calc software. p<0.05.Considering the overall 2-10 fold GFP+% increase in the primary transplants, miR-125a has expanded hematopoietic stem cell numbers by an estimated 6-30 fold. Of note, the increase in stem cells was not associated with complete differentiation blockade (though there was an increase in myeloid cells) and no animals developed evidence of leukemia.

MiR-125a Protected Primitive Hematopoietic Cells from Apoptosis

Lastly, since Dicer loss induced apoptosis in the primitive multipotent hematopoietic compartment, it was asked whether expression of miR-125a may protect primitive cells from apoptosis. Whole bone marrow from transplanted animals was stained with lineage cocktail, AnnexinV and 7-AAD. 7-AAD-GFP+ cells were divided into two populations based on their expression of collective lineage markers. Lineage negative population is defined as the lowest 3% cells expressing lineage markers. Bone marrow from mice transplanted with control- or miR-125a-transduced cells were analyzed for apoptosis after re-establishment of homeostasis. It was observed that there was decreased apoptosis in miR-125a-tranduced cells in the lineage-negative population, but not in the more mature lineage-positive cells (FIG. 4I), indicating a cell-type-specific effect of miR-125a. These data are consistent with the anti-apoptotic effect being at least partially responsible for the increase in the reconstituting cell pool.

### MiR-125a Targets Bak1

The HSC pool size is controlled by balancing fates of self-renewal versus differentiation and apoptosis. HL-60 cells were infected with a vector control virus (Control) or one that expresses miR-125a. Total protein was resolved by SDS-PAGE and probed for Bak1, or β-actin as a loading control. Indeed, over-expression of Bcl-2 has been shown to increase the stem cell pool. Given the reduced apoptosis in primitive cells and the greatly expanded HSC pool size, it was reasonable that miR-125a could target certain pro-apoptotic proteins. Among the predicted evolutionarily conserved miR-125a targets, Bak1 (Bcl-2 antagonist/killer 1) is well known for its pro-apoptotic activity. HL-60 cells were transduced with a control virus or one that expresses miR-125a. MiR-125a expression reduced endogenous Bak1 protein by ˜50% (FIG. 4J). Scanning the 3′ untranslated region (UTR) of Bak1 revealed one conserved miR-125a targeting site. To ascertain whether the inhibitory effect of miR-125a was mediated through its 3′UTR, the UTR sequence was fused to a luciferase reporter. The Bak1 3′UTR sequence, wild type (WT) or mutant for the miR-125a target site, were cloned 3′ to a luciferase reporter. These reporter constructs were co-transfected into 293T cells in triplicates with either a control vector or miR-125a. MiR-125a caused ˜50% inhibition of the luciferase activity. In addition, mutation of the conserved miR-125a targeting site alleviated most of the inhibition by miR-125a (FIG. 4K). Consistent with the observation that miR-125a is the single microRNA within the miR-99b-let-7e-miR-125a cluster that mediates the HSC expansion; miR-125a inhibited the Bak1 3′UTR construct (FIG. 4L), while miR-99b and let-7e had minimal effect. Taken together, out data are consistent with miR-125a antagonizing a critical pro-apoptotic protein to relieve HSPCs from apoptosis allowing extensive expansion of hematopoietic stem cells.

Reported herein is an essential role of Dicer for HSC maintenance and identified a single microRNA, miR-125a, capable of positively regulating HSC regeneration of hematopoiesis. In other systems where Dicer was deleted, e.g. embryonic stem cells, T and B cells, cells tolerated a Dicer null status and did not die, albeit proliferation and differentiation abnormalities were often observed. HSCs however, display absolute dependence on Dicer for maintenance. In the absence of Dicer, stem cells do not have continued ability to produce blood cells, as shown by the much reduced levels immunophenotypically and undergo apoptosis with increased frequency. Stem cells usually possess multiple fate options and adoption of a determined fate is associated with extensive epigenetic reinforcement that may involve microRNAs. Lack of microRNAs may severely compromise the fate choice required for proper stem cell function, but perhaps be less critical for cells where lineage commitment has occurred and more durable epigenetic mechanisms of cell state have been imposed.

Given the relative increase in HSC and the improved ability to serially transplant irradiated recipients, the data strongly indicates that miR-125a is a regulator of the signature stem cell function, self-renewal.

TABLE 1
Before plpC

| | WBC | NE | LY | MO | EO | BA | platelets | RBC |
|---|---|---|---|---|---|---|---|---|
| Control | 10.55 ± 2.67 | 1.12 ± 0.56 | 8.59 ± 2.04 | 0.76 ± 0.14 | 0.06 ± 0.04 | 0.01 ± 0.02 | 481 ± 48.48 | 8.76 ± 0.98 |
| Mutant | 10.45 ± 3.23 | 1.47 ± 0.84 | 7.90 ± 2.19 | 0.85 ± 0.22 | 0.19 ± 0.25 | 0.05 ± 0.09 | 405.4 ± 97.49 | 9.09 ± 1.31 |
| p value | 0.945 | 0.300 | 0.500 | 0.433 | 0.140 | 0.251 | 0.665 | 0.581 |

TABLE 2
One day after plpC.

| | WBC | NE | LY | MO | EO | BA | platelets | RBC |
|---|---|---|---|---|---|---|---|---|
| Control | 12.46 ± 2.60 | 2.24 ± 0.58 | 9.19 ± 2.04 | 0.55 ± 0.17 | 0.40 ± 0.13 | 0.09 ± 0.03 | 411.8 ± 75.8 | 11.05 ± 1.11 |
| Mutant | 6.02 ± 0.81 | 0.94 ± 0.36 | 4.65 ± 0.49 | 0.24 ± 0.07 | 0.15 ± 0.03 | 0.04 ± 0.01 | 216 ± 138.9 | 9.88 ± 1.10 |
| p value | 0.003 | 0.004 | 0.006 | 0.011 | 0.012 | 0.030 | 0.032 | 6.132 |

TABLE 3
Three weeks after plpC.

| | WBC | NE | LY | MO | EO | BA | platelets | RBC |
|---|---|---|---|---|---|---|---|---|
| Control | 8.31 ± 2.12 | 1.75 ± 0.84 | 5.74 ± 1.69 | 0.35 ± 0.05 | 0.36 ± 0.13 | 0.10 ± 0.04 | 336 ± 54.38 | 10.07 ± 1.79 |
| Mutant | 8.3 ± 2.48 | 1.87 ± 0.92 | 5.72 ± 1.52 | 0.42 ± 0.14 | 0.24 ± 0.08 | 0.06 ± 0.03 | 297.8 ± 112.6 | 9.21 ± 1.47 |
| p value | 0.998 | 0.654 | 0.985 | 0.445 | 0.169 | 0.141 | 0.552 | 0.487 |

Table 4: Top markers that distinguish LT vs. others. Normalized based on RNA quantity.

TABLE 5

| | Input Cell genotype | | |
|---|---|---|---|
| Colony Genotype | lox/lox | lox/wt | wt/wt |
| Δ/Δ | 0/75 | — | — |
| lox/lox | 23/75 | — | — |
| lox/Δ | 52/75 | — | — |
| Δ/wt | — | 8/8 | — |
| lox/wt | — | 0/8 | — |
| wt/wt | — | — | 8/8 |

1 day after the 7^{th} plpC injection in non transplanted animals, WBM were plated into m3434.
After 2 weeks, colonies were plucked and tested for Diecer delection.

### Listing of Sequences:

(SEQ. ID. No. 1)
Homo sapiens, pre-miR-125a, Stem-loop sequence
MI0000469
MiRBase Accession No. MI0000469 (at the Microrna
Database at Sanger UK)
ID: hsa-mir-125a
Symbol: HGNC:MIRN125A
5′UGCCAGUCUCUAGGUCCCUGAGACCCUUUAACCUGUGAGGACAUCC
AGGGUCACAGGUGAGGUUCUUGGGAGCCUGGCGUCUGGCC-3′
(SEQ. ID. No. 2)
Homo sapiens mature miR-125a sequence
MIMAT0000443
MiRBase Accession No. MIMAT0000443
ID: hsa-miR-125a-5p
UCCCUGAGACCCUUUAACCUGUGA
(SEQ. ID. No. 3)
Homo sapiens minor miR-125a sequence
MIMAT0004602 (imperfect complementary strand to
the mature miR-125a
MiRBase Accession No. MIMAT0004602
ID: hsa-miR-125a-3p
ACAGGUGAGGUUCUUGGGAGCC

1. A method of expanding ex vivo a population of hematopoietic stem cells (HSCs), the method comprising contacting an isolated HSC with a nucleic acid sequence comprising miR-125a, thereby expanding ex vivo a population of HSCs.

2. The method of claim 1, wherein the HSC is isolated from peripheral blood, umbilical cord blood, or bone marrow from a subject.

3. The method of claim 1, wherein the nucleic acid sequence comprises SEQ. ID. No. 1 or 2.

4-7. (canceled)

8. The method of claim 1, further comprising expanding the HSCs for at least one cell doubling ex vivo.

9. The method of claim 8, further comprising cryopreserving the expanded HSCs.

10-40. (canceled)

41. A method of expanding hematopoietic stem cell (HSC) production in a subject in need thereof, the method comprising providing a therapeutically effective amount of a nucleic acid sequence comprising miR-125a to the subject, thereby expanding HSC production in the subject.

42. The method of claim 41, wherein the nucleic acid sequence comprising miR-125a comprises SEQ. ID. No. 1 or 2.

43. (canceled)

44. The method of claim 41, wherein the therapeutically effective amount of a nucleic acid sequence comprising miR-125a is provided by administering a pharmaceutical composition comprising i) a nucleic acid sequence comprising miR-125a or ii) a vector expressing a nucleic acid sequence encoding miR-125a or a precursor thereof.

45-49. (canceled)

50. The method of claim 41, wherein the subject has received, will receive or is concurrently receiving chemotherapy or radiation therapy.

51. The method of claim 50, wherein the subject has a disorder selected from the group consisting of myeloma, non-Hodgkin's lymphoma, Hodgkins lyphoma and leukaemia.

52. The method of claim 51, wherein the subject has received, will receive or is concurrently receiving granulocyte colony-stimulating factor (G-CSF).

53. The method of claim 41, wherein the subject has a disorder characterized by a lack of functional blood cells.

54-57. (canceled)

58. The method of claim 41, wherein the subject has a disorder characterized by a lack of functional immune cells.

59. The method of claim 58, wherein the immune cells are T or B lymphocytes.

60. (canceled)

61. The method of claim 41, wherein the subject has received, will receive or is receiving an immuno-suppressive drug.

62. The method of claim 41, wherein the subject is a stem cell donor.

63. (canceled)

64. A method of expanding HSC production in a subject in need thereof, the method comprising providing a therapeutically effective amount of an agent that increases the expression miR-125a to the subject, thereby expanding HSC production in the subject.

65. The method of claim 64, wherein the agent is selected from a group consisting of a small molecule, nucleic acid, nucleic acid analogue, protein, antibody, peptide, aptamer and variants or fragments thereof.

66. The method of claim 64, wherein the therapeutically effective amount of an agent is provided by administering a pharmaceutical composition comprising the agent and a pharmaceutically acceptable carrier.

67. The method of claim 10, wherein the expanded HSC are administered to a subject in a pharmaceutical composition comprising the HSC and a pharmaceutically acceptable carrier.

68-70. (canceled)
